(19) United States
(12) Patent Application Publication
(10) Pub. No.: US 2023/0321055 A1
(43) Pub. Date: Oct. 12, 2023

(54) PREVENTIVE TREATMENT OF MIGRAINE

(71) Applicant: Allergan Pharmaceuticals International Limited, Dublin (IE)

(72) Inventors: Joel Trugman, Hoboken, NJ (US); Michelle Finnegan, New York, NY (US)

(21) Appl. No.: 18/181,228

(22) Filed: Mar. 9, 2023

Related U.S. Application Data
(60) Provisional application No. 63/480,365, filed on Jan. 18, 2023.
(60) Provisional application No. 63/404,352, filed on Sep. 7, 2022.
(60) Provisional application No. 63/347,265, filed on May 31, 2022.
(60) Provisional application No. 63/269,105, filed on Mar. 9, 2022.

(51) Int. Cl. A61K 31/438; A61P 25/04

(52) U.S. Cl. CPC A61K 31/438; A61P 25/04

(57) ABSTRACT

The present disclosure provides medicaments and methods for the preventive treatment of migraine, particularly the preventive or prophylactic treatment of chronic migraine.

### CROSS-REFERENCE TO RELATED APPLICATIONS

This application claims the benefit of priority to U.S. Provisional Application No. 63/269,105, filed Mar. 9, 2022; U.S. Provisional Application No. 63/347,265, filed May 31, 2022; U.S. Provisional Application No. 63/404,352, filed Sep. 7, 2022; and U.S. Provisional Application No. 63/480,365, filed Jan. 18, 2023. The contents of each of these applications are incorporated herein by reference in their entirety.

### FIELD

The present disclosure is related to medicaments and methods for treating migraine, particularly the preventive or prophylactic treatment of chronic migraine.

### BACKGROUND

Migraine is a highly prevalent, severe, and disabling neurological condition with a significant unmet need for effective treatments. (Holland, P. R. & Goadsby, P. J. Neurotherapeutics (2018)). Migraine affects over 1 billion people worldwide, and it was reported as the second leading cause of disability in the 2016 Global Burden of Disease study. See GBD 2019 Diseases and Injuries Collaborators. Global Burden of 369 diseases and injuries in 204 countries and territories, 1990-2019: a systemic analysis for the Global Burden of Disease Study 2019, Lancet 2020; 396:1204-22.

Chronic migraine represents a particularly debilitating type of migraine. Chronic migraine can be severely disabling and difficult to manage. Relative to patients with episodic migraine, patients with chronic migraine experience much more frequent headaches, comorbid pain and affective disorders, and fewer pain-free intervals. Diener et al., “Chronic migraine—classification, characteristics and treatment.” Nat Rev Neurol. 2012 Feb. 14;8(3):162-71; see also Agosti, “Migraine Burden of Disease: From the Patient's Experience to a Socio-Economic View”, Headache: The Journal of Head and Face Pain, 58: 17-32 (2018).

Current preventive treatments for chronic migraine include onabotulinumtoxinA, topiramate, or monoclonal antibodies targeting calcitonin gene-related peptide (CGRP). There remains a need for methodologies and dosing regimens for oral CGRP therapies for the preventive treatment of chronic migraine.

### SUMMARY

In embodiments, the present disclosure provides a method for the preventive treatment of chronic migraine, the method comprising administering atogepant or a pharmaceutically acceptable salt thereof to a patient in need thereof. In embodiments, atogepant is administered in an amount of 30 mg twice daily. In embodiments, atogepant is administered in an amount of 60 mg once daily.

In embodiments, the present disclosure provides a method of statistically significant treatment of migraine in patients in need thereof, wherein the migraine is chronic migraine. In embodiments, the method comprises administering to each patient atogepant or a pharmaceutically acceptable salt thereof in an amount of 30 mg twice daily or 60 mg once daily, wherein said treatment achieves a statistically significant least square mean difference in monthly migraine days in said patients from baseline to week 12 of said treatment as compared to placebo. In embodiments, treatment with atogepant also achieves a statistically significant improvement in one or more of the following:
(a) change from baseline in mean monthly headache days;
(b) change from baseline in mean monthly acute medication use days;
(c) proportion of patients achieving at least a 50% reduction in 3-month average of monthly migraine days;
(d) change from baseline in MSQ v2.1 Role Function-Restrictive Domain Score;
(e) change from baseline in Mean Monthly Performance of Daily Activities Domain Score of the Activity Impairment in Migraine-Diary (AIM-D);
(f) change from baseline in the Mean Monthly Physical Impairment Domain Score of the AIM-D; or
(g) change from baseline in HIT-6 (Headache Impact Test) total score.

In embodiments, the present disclosure provides a method for the preventive treatment of chronic migraine, the method comprising administering atogepant or a pharmaceutically acceptable salt thereof in an amount of 30 mg twice daily or 60 mg once daily to a patient in need thereof, wherein treatment with atogepant achieves a statistically significant reduction from baseline in mean monthly migraine days. In embodiments, treatment with atogepant also achieves a statistically significant improvement in one or more of the following:
(h) change from baseline in mean monthly headache days;
(i) change from baseline in mean monthly acute medication use days;
(j) proportion of patients achieving at least a 50% reduction in 3-month average of monthly migraine days;
(k) change from baseline in MSQ v2.1 Role Function-Restrictive Domain Score;
(l) change from baseline in Mean Monthly Performance of Daily Activities Domain Score of the Activity Impairment in Migraine-Diary (AIM-D);
(m)change from baseline in the Mean Monthly Physical Impairment Domain Score of the AIM-D; or
(n) change from baseline in HIT-6 (Headache Impact Test) total score.

### BRIEF DESCRIPTION OF THE FIGURES

FIG. 1 provides a schematic of the ph. 3, multicenter, randomized, double-blind, placebo-controlled, parallel group study carried out to evaluate the efficacy, safety, and tolerability of atogepant for the prevention of chronic migraine.

FIG. 2A shows the least square mean (+/−SE) of change from baseline in monthly migraine days (MMRM) during the double-blind treatment period in the Modified Intent-to-Treat (mITT) population. Both atogepant doses (30 mg twice daily and 60 mg once daily) demonstrated a statistical and clinical improvement in change from baseline in monthly migraine days, the primary efficacy endpoint. FIG. 2B shows the distribution of change from baseline in mean MMD across the 12-week treatment period, in 2-day increments, by treatment group. A treatment benefit of atogepant 60 mg over placebo is seen across a range of mean changes from baseline in MMD.

FIG. 3 shows the least square mean (+/−SE) of change from baseline in monthly headache days (MMRM) during the double-blind treatment period for the mITT population. The analysis demonstrated that treatment with atogepant 60 mg once daily and 30 mg twice daily resulted in statistically and clinically significant improvement in monthly headache days.

FIG. 4 shows the least square mean (+/−SE) of change from baseline in acute medication use days (MMRM) during the double blind treatment period for the mITT population. The analysis demonstrated that treatment with atogepant 60 mg once daily and 30 mg twice daily resulted in statistically and clinically significant improvement in acute medication use days.

FIG. 5 shows the cumulative distribution function of percent reduction from baseline in 3-month average of monthly migraine days in the mITT population. The analysis demonstrated that treatment with atogepant 60 mg once daily and 30 mg twice daily resulted in statistically and clinically significant improvement in 3-month average of monthly migraine days.

FIG. 6 shows mean changes from baseline in monthly migraine days and weekly migraine days (inset) during weeks 1-4 for atogepant 30 mg BID and atogepant 60 mg QD vs. placebo. In adults with CM, atogepant demonstrated an early and sustained reduction in migraine days. Results showed a statistically significant effect of treatment during each week of the first 4-week intervals, and as early as the first full day after study drug initiation.

FIG. 7A, FIG. 7B, FIG. 7C, and FIG. 7D show the proportion of patients treated with atogepant 30 mg BID, atogepant 60 mg QD, and placebo who experienced a ≥30%, ≥50%, ≥75%, or 100% reduction in MMDs at weeks 1-4, weeks 5-8, and weeks 9-12. Both atogepant dosing regimens increased the proportions of participants with CM achieving ≥30%, ≥50%, ≥75%, or 100% reduction in mean MMDs across 12 weeks. A higher proportion of atogepant-treated participants with CM reported overall improvement on the PGI-C and in treatment satisfaction.

FIG. 8 shows the change from baseline in AIM-D Performance of Daily Activities at Weeks 1-4, 5-8, 9-12 and across 12 weeks. Treatment with atogepant 60 mg once daily and 30 mg twice daily demonstrated statistically significant improvements from baseline across the 12-week treatment period in AIM-D PDA (least squares mean difference [LSMD]: 30mg BID, −4.85 [95% CI: −6.75, −2.95]; 60 mg QD, −3.38 [−5.27, −1.49]; P<0.001) and PI (LSMD: 30 mg BID, −4.19 [95% CI: −5.95, −2.43]; 60 mg QD, −2.71 [−4.47, −0.96]; P<0.01) domain scores vs placebo.

FIG. 9 shows the change from baseline in WAPI Overall Work Productivity Loss at Weeks 1-4, 5-8, and 9-12. Both doses of atogepant (30 mg BID and 60 mg QD) demonstrated nominally significant improvements in presenteeism, overall work productivity loss, and activity impairment for both doses at all time points, and in absenteeism at weeks 4 and 12 for both doses, vs. placebo (p<0.05).

FIG. 10 shows the change from baseline in MSQ Role Function-Restrictive Domain at Weeks 4, 8, and 12. Increases from baseline (improvements) in a three MSQ domain scores were significantly greater in both atogepant treatment arms (30 mg BID and 60 mg QD) compared with placebo.

FIG. 11 shows the change from baseline in mean MMDs across the 12-week treatment period in participants with and without acute medication overuse headache. Among those with acute medication overuse at baseline, the least squares mean (LSM) change from baseline in mean MMDs in atogepant-treated participants (30 mg BID and 60 mg QD) was −8.3 and −7.5 compared with −5.6 in the placebo arm. Regardless of acute medication overuse at baseline, atogepant was shown to be effective and was associated with a greater reduction in acute medication use days in people with chronic migraine.

FIG. 12 shows data for patients treated with atogepant 30 mg BID, atogepant 60 mg QD, and placebo who experienced a decrease from baseline in their mean weekly migraine days during the first month of administration.

FIG. 13 shows the proportion (%) of patients treated with atogepant 30 mg BID, atogepant 60 mg QD, and placebo who experienced a migraine day during the first week of treatment.

FIG. 14 provides a revised schematic of a phase 3, multicenter, randomized, double-blind, placebo-controlled, parallel group study carried out to evaluate the efficacy, safety, and tolerability of atogepant for the prevention of chronic migraine.

FIG. 15 shows the change from baseline in WPAI Presenteeism at Weeks 1-4, 5-8, and 9-12. Both doses of atogepant (30 mg BID and 60 mg QD) demonstrated nominally significant improvements in presenteeism, overall work productivity loss, and activity impairment for both doses at all time points, and in absenteeism at weeks 4 and 12 for both doses, vs. placebo (p<0.05).

FIG. 16 shows the change from baseline in AIM-D Physical Impairment at Weeks 1-4, 5-8, 9-12 and across 12 weeks. Treatment with atogepant 60 mg once daily and 30 mg twice daily demonstrated statistically significant improvements (i.e., reductions) from baseline across the 12-week treatment period in AIM-D PI (least squares mean difference [LSMD].

FIG. 17 shows the change from baseline in WPAI Absenteeism at Weeks 1-4, 5-8, and 9-12. Both doses of atogepant (30 mg BID and 60 mg QD) demonstrated nominally significant improvements in presenteeism, overall work productivity loss, and activity impairment for both doses at all time points, and in absenteeism at weeks 4 and 12 for both doses, vs. placebo (p<0.05).

FIG. 18 shows the change from baseline in WPAI activity impairment at Weeks 1-4, 5-8, and 9-12. Both doses of atogepant (30 mg BID and 60 mg QD) demonstrated nominally significant improvements in presenteeism, overall work productivity loss, and activity impairment for both doses at all time points, and in absenteeism at weeks 4 and 12 for both doses, vs. placebo (p<0.05).

FIG. 19 provides a chart describing participant disposition in each stage of the ph. 3, multicenter, randomized, double-blind, placebo-controlled, parallel group study carried out to evaluate the efficacy, safety, and tolerability of atogepant for the prevention of chronic migraine.

FIG. 20 shows the change from baseline in MSQ Emotional Function-Domain at Weeks 4, 8, and 12.

FIG. 21 shows the change from baseline in MSQ Role Function-Preventative Domain at Weeks 4, 8, and 12.

FIG. 22 shows the change from baseline in Headache Impact Test (HIT-6) scores for both doses of atogepant (30 mg BID and 60 mg QD) vs placebo at Weeks 4, 8, and 12. Both doses demonstrated a statistically significant improvement vs placebo at week 12 (P<0.001), as well as nominally significant improvements vs placebo at weeks 4 and 8 (nominal P<0.001).

FIG. 23 shows the sustained response in months 2-3 among participants with an initial response of ≥30% or ≥50% reduction in mean MMDs.

FIG. 24 shows percentage of initial nonresponders who experienced a ≥30% or ≥50% reduction in mean MMDs in subsequent months.

FIG. 25A and FIG. 25B show the change from baseline in AIM-D (A) performance of daily activities and (B) physical impairment at weeks 1, 2, 3, and 4.

FIG. 26A and FIG. 26B show change from baseline in EQ-5D-5L (A) descriptive system index and (B) visual analogue scale scores at weeks 1-2 and week 4.

### DETAILED DESCRIPTION

Migraine, a debilitating disorder that impacts 14% of the population, can be subtyped as episodic migraine (EM) or chronic migraine (CM). Chronic migraine is a debilitating neurological disease where patients experience headaches occurring on 15 or more days per month for more than three months, which on at least eight days per month has features of migraine headaches. Episodic migraine is characterized by headaches that occur on fewer than 15 days per month. Lipton et al., “Episodic and chronic migraine headache: breaking down barriers to optimal treatment and prevention”, Headache, 2015 March;55 Suppl 2:103-22; quiz 123-6; Headache Classification Committee., Olesen J, Bousser M G, Diener H C, Dodick D, First M, Goadsby P J, Göbel H, Lainez M J, Lance J W, Lipton R B, Nappi G, Sakai F, Schoenen J, Silberstein S D, Steiner T J Cephalalgia. 2006 June; 26(6):742-6.

Beyond the 15-day threshold, chronic migraine is distinguished from episodic migraine in both burden of illness and the treatment needs of patients. See Ishii et al., “Chronic versus episodic migraine: The 15-day threshold does not adequately reflect substantial differences in disability across the full spectrum of headache frequency.” Headache, 2021 July; 61(7):992-1003. Chronic migraine is particularly debilitating and difficult to manage. Clinical and population-based studies have demonstrated that chronic migraine results in greater migraine-related disability and impairment in headache related quality of life than episodic migraine. Chronic migraine is associated with greater incidence of comorbidities, higher indirect and indirect costs, and different patterns of consultation and treatment than episodic migraine. Buse et al., “Sociodemographic and comorbidity profiles of chronic migraine and episodic migraine sufferers”, Journal of Neurology, Neurosurgery & Psychiatry 2010;81:428-432; Mungoven et al., “Chronic Migraine Pathophysiology and Treatment: A Review of Current Perspectives.” Front. Pain Res., 25 Aug. 2021.

In embodiments, the present disclosure provides methods for the prophylactic or preventive treatment of chronic migraine. In embodiments, the present disclosure provides methods for the preventive treatment of migraine, such as the preventive treatment of chronic migraine, comprising orally administering an effective amount of an oral CGRP receptor antagonist. In embodiments, the CGRP receptor antagonist is atogepant. The chemical name of atogepant is (S)—N—((3S,5S,6R)-6-methyl-2-oxo-1-(2,2,2-trifluoroethyl)-5-(2,3,6-trifluorophenyl)piperidin-3-yl)-2′-oxo-1′,2′,5,7- tetrahydrospiro[cyclopenta[b]pyridine-6,3′-pyrrolo[2,3-b]pyridine]-3-carboxamide and has the following structural formula:

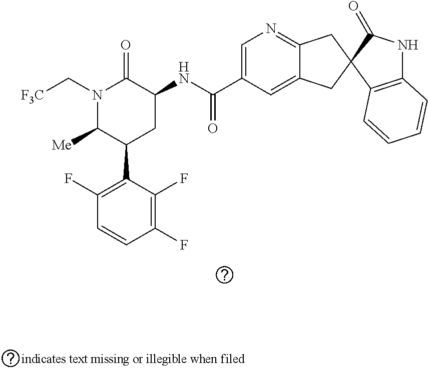 [SMILES: C[C@@H]1[C@H](c2c(F)ccc(F)c2F)C[C@H](NC(=O)c2cnc3c(c2)C[C@@]2(C3)C(=O)Nc3ccccc32)C(=O)N1CC(F)(F)F]

The molecular formula is C₂₉H₂₃F₆N₅O₃ and molecular weight is 603.5. Atogepant is a small molecule calcitonin gene-related peptide (CGRP) receptor antagonist that blocks the binding of the CGRP to the receptor and antagonizes CGRP receptor function. Atogepant may be administered orally, reaching maximum plasma concentrations by 2 hours, with a half-life of approximately 11 hours.

In embodiments, a patient in need of treatment for chronic migraine meets the definition of chronic migraine set forth in the International Classification of Headache Disorders, 3rd edition (ICHD-3).

Prophylactic or preventive treatment can reduce the frequency and/or intensity of migraine attacks. In embodiments, the preventive or prophylactic treatment methods of the present disclosure can reduce the frequency and/or intensity of migraine attacks. In embodiments, the preventive or prophylactic treatment methods of the present disclosure can reduce the frequency and/or intensity of symptoms associated with migraine attacks, including headaches. In embodiments, the administration of atogepant may provide for fewer symptoms of migraine or symptoms of reduced intensity. In embodiments, the non-headache symptoms of migraine may be reduced or eliminated. In embodiments, the prophylactic methods of the present disclosure can result in freedom from symptoms associated with migraine attacks, including headaches. In embodiments, the prophylactic methods of the present disclosure are directed to the entire range of symptoms experienced by a patient during a migraine attack, and not solely at the prevention of headaches associated with a migraine attack.

In embodiments, a patient in need of treatment for chronic migraine may suffer from one or more symptoms of migraine including, for example, sinusitis, nausea, nasopharyngitis, photophobia, appetite changes, cognition and concentration difficulties, cold extremities, diarrhea or other bowel changes, excitement or irritability, fatigue, frequent urination, memory changes, weakness, yawning, stretching, seeing bright spots or flashes of light, vision loss, seeing dark spots, tingling sensations, speech problems, aphasia, tinnitus, gastric stasis, pulsating or throbbing pain on one or both sides of the head, extreme sensitivity to light, sounds, or smells, worsening pain during physical activity, and vomiting, abdominal pain or heartburn, loss of appetite, lightheadedness, blurred vision, and fainting. In embodiments, the administration of a prophylactically effective amount of atogepant results in the improvement, reduced frequency, or reduced intensity of symptoms.

In embodiments, the present disclosure provides a method for the prophylactic or preventive treatment of chronic migraine, the method comprising administering to a patient in need thereof a therapeutically effective amount of atogepant, or a pharmaceutically acceptable salt thereof. In embodiments, atogepant is administered orally at a once-daily (QD) dose of 60 mg or a twice-daily (BID) dose of 30 mg. In embodiments, atogepant is administered orally at a once-daily (QD) dose of 60 mg. In embodiments, atogepant is administered orally at a twice-daily (BID) dose of 30 mg.

In embodiments, atogepant is administered orally at a once-daily dose of 60 mg for at least about one week, or at least about 2 weeks, or at least about 3 weeks, or at least about 4 weeks, or at least about 8 weeks, or at least about 12 weeks, or at least about 16 weeks, or at least about 20 weeks, or at least about 24 weeks, or at least about 28 weeks, or at least about 32 weeks, or at least about 36 weeks, or at least about 40 weeks, or at least about 44 weeks, or at least about 48 weeks, or at least about 52 weeks. In embodiments, atogepant is administered for at least about one month, or at least about two months, or at least about three months.

In embodiments, atogepant is administered orally at a twice-daily (i.e., BID) dose of 30 mg for at least about one week, or at least about 2 weeks, or at least about 3 weeks, or at least about 4 weeks, or at least about 8 weeks, or at least about 12 weeks, or at least about 16 weeks, or at least about 20 weeks, or at least about 24 weeks, or at least about 28 weeks, or at least about 32 weeks, or at least about 36 weeks, or at least about 40 weeks, or at least about 44 weeks, or at least about 48 weeks, or at least about 52 weeks. In embodiments, atogepant is administered for at least about one month, or at least about two months, or at least about three months.

In embodiments, treatment with atogepant 30 mg BID or 60 mg QD for the preventive treatment of chronic migraine achieves a statistically significant change from baseline in mean monthly migraine days. In embodiments, treatment with atogepant achieves a statistically significant change from baseline in mean monthly headache days. In embodiments, treatment with atogepant achieves at least a 50% reduction in 3-month average of monthly migraine days in a statistically significant proportion of subjects. In embodiments, treatment with atogepant achieves a statistically significant change from baseline in MSQ v2.1 Role Function-Restrictive Domain Score. In embodiments, treatment with atogepant achieves a statistically significant change from baseline in Mean Monthly Performance of Daily Activities Domain Score of the AIM-D. In embodiments, treatment with atogepant achieves a statistically significant change from baseline in Mean Monthly Physical Impairment Domain Score of the AIM-D. In embodiments, treatment with atogepant achieves a statistically significant change from baseline in HIT-6 total score.

In embodiments, in a 12-week treatment period, treatment with atogepant 30 mg BID or 60 mg QD for the preventive treatment of chronic migraine achieves a statistically significant reduction from baseline in mean monthly migraine days across the 12-week treatment period, a statistically significant change from baseline in mean monthly acute medication use days across the 12-week treatment period, a statistically significant proportion of patients achieving at least a 50% reduction in 3-month average of monthly migraine days, a statistically significant change from baseline in MSQ v2.1 Role Function-Restrictive Domain Score at Week 12, a statistically significant change from baseline in the Mean Monthly Performance of Daily Activities Domain Score of the AIM-D across the 12-week treatment period, and a statistically significant change from baseline in the Mean Monthly Physical Impairment Domain Score of the AIM-D across the 12-week treatment period.

In embodiments, in a 12-week treatment period, treatment with atogepant 30 mg BID or 60 mg QD for the preventive treatment of chronic migraine achieves a statistically significant reduction from baseline in mean monthly migraine days across the 12-week treatment period, a statistically significant change from baseline in mean monthly acute medication use days across the 12-week treatment period, a statistically significant proportion of patients achieving at least a 50% reduction in 3-month average of monthly migraine days, a statistically significant change from baseline in HIT-6 total score at week 12, and a statistically significant change from baseline in MSQ v2.1 Role Function-Restrictive Domain Score at Week 12.

In embodiments, the present disclosure provides a method for the preventive treatment of migraine, such as the preventive treatment of chronic migraine, the method comprising administering atogepant in an amount of 60 mg once daily or 30 mg twice daily to a patient or patients in need thereof, wherein treatment with atogepant achieves a reduction from baseline in mean monthly migraine days (MMDs). In embodiments, treatment with atogepant achieves a reduction from baseline in mean monthly migraine days of at least about 6 days, or at least about 6.2 days, or at least about 6.4 days, or at least about 6.8 days, or at least about 7 days, or at least about 7.2 days, or at least about 7.4 days.

In embodiments, the present disclosure provides a method for the preventive treatment of migraine in patients in need thereof, wherein the migraine is chronic migraine, comprising administering to each said patient atogepant or a pharmaceutically acceptable salt thereof in an amount of 60 mg once daily or 30 mg twice daily, and said treatment achieves statistically significant least square mean difference in monthly migraine days (MMDs) in said patients from baseline of said treatment compared to placebo. In embodiments, treatment with atogepant achieves a least square mean difference in mean monthly migraine days from baseline to week 12 of said treatment of at least about −1, or at least about −1.2, or at least about −1.4, or at least about −1.6, or at least about −1.8, or at least about −2, or at least about −2.2, or at least about −2.4 as compared to placebo. In embodiments, treatment with atogepant achieves a statistically significant improvement from baseline in migraine days within the first week of treatment, or within the first two weeks of treatment, or within the first three weeks of treatment, or within the first four weeks of treatment. In embodiments, treatment with atogepant achieves an improvement from baseline as early as the first full day after initiating treatment with atogepant.

In embodiments, the present disclosure provides a method for the preventive treatment of migraine, wherein the migraine is chronic migraine, the method comprising administering atogepant in an amount of 30 mg twice daily to a patient or patients in need thereof, resulting in a reduction in mean monthly migraine days. In embodiments, treatment with atogepant 30 mg twice daily achieves a reduction from baseline in mean monthly migraine days of at least about 6 days, or at least about 6.2 days, or at least about 6.4 days, or at least about 6.8 days, or at least about 7 days, or at least about 7.2 days, or at least about 7.4 days. In embodiments, the present disclosure provides a method for the preventive treatment of chronic migraine, the method comprising administering atogepant in an amount of 30 mg twice daily to a patient or patients in need thereof, wherein treatment with atogepant results in a reduction from baseline in mean monthly migraine days of about 7.3 days across a 12-week treatment period. In embodiments, the present disclosure provides a method for the preventive treatment of chronic migraine, the method comprising administering atogepant in an amount of 30 mg twice daily to a patient or patients in need thereof, wherein treatment with atogepant achieves a reduction from baseline in mean monthly migraine days of about 7.4 days across a 12-week treatment period. In embodiments, treatment with atogepant achieves a statistically significant improvement from baseline in migraine days within the first week of treatment, or within the first two weeks of treatment, or within the first three weeks of treatment, or within the first four weeks of treatment. In embodiments, treatment with atogepant achieves an improvement from baseline as early as the first full day after initiating treatment with atogepant.

In embodiments, the present disclosure provides a method for the preventive treatment of migraine in patients in need thereof, wherein the migraine is chronic migraine, comprising administering to said patients atogepant or a pharmaceutically acceptable salt thereof in an amount of 30 mg twice daily, and wherein said treatment achieves statistically significant least square mean difference in mean monthly migraine days in said patients from baseline of said treatment compared to placebo. In embodiments, treatment with atogepant achieves a least square mean difference in mean monthly migraine days from baseline to week 12 of said treatment of at least about −1, or at least about −1.2, or at least about −1.4, or at least about −1.6, or at least about −1.8, or at least about −2, or at least about −2.2, or at least about −2.4 as compared to placebo. In embodiments, treatment with atogepant achieves a least square mean difference in mean monthly migraine days from baseline to week 12 of said treatment of at least about −2.2 as compared to placebo. In embodiments, treatment with atogepant achieves a least square mean difference in mean monthly migraine days from baseline to week 12 of said treatment of at least about −2.4 as compared to placebo across a 12-week treatment period. In embodiments, treatment with atogepant achieves a statistically significant improvement from baseline in migraine days within the first week of treatment, or within the first two weeks of treatment, or within the first three weeks of treatment, or within the first four weeks of treatment. In embodiments, treatment with atogepant achieves an improvement from baseline as early as the first full day after initiating treatment with atogepant.

In embodiments, the present disclosure provides a method for the preventive treatment of migraine, wherein the migraine is chronic migraine, comprising administering atogepant in an amount of 60 mg QD to a patient or patients in need thereof, resulting in a reduction in mean monthly migraine days. In embodiments, treatment with atogepant 60 mg QD achieves a reduction from baseline in mean monthly migraine days of at least 6 days, or at least about 6.2 days, or at least about 6.4 days, or at least about 6.8 days. In embodiments, the present disclosure provides a method for the preventive treatment of migraine, wherein the migraine is chronic migraine, comprising administering atogepant in an amount of 60 mg once daily to a patient or patients in need thereof, wherein treatment with atogepant achieves a reduction from baseline in mean monthly migraine days of about 6.7 days across a 12-week treatment period. In embodiments, the present disclosure provides a method for the preventive treatment of migraine, wherein the migraine is chronic migraine, comprising administering atogepant in an amount of 60 mg QD to a patient or patients in need thereof, wherein treatment with atogepant achieves a reduction from baseline in mean monthly migraine days of about 6.8 days across a 12-week treatment period. In embodiments, treatment with atogepant achieves a statistically significant improvement from baseline in migraine days within the first week of treatment, or within the first two weeks of treatment, or within the first three weeks of treatment, or within the first four weeks of treatment. In embodiments, treatment with atogepant achieves an improvement from baseline as early as the first full day after initiating treatment with atogepant.

In embodiments, the present disclosure provides a method for the preventive treatment of migraine in patients in need thereof, wherein the migraine is chronic migraine, comprising administering to said patients atogepant or a pharmaceutically acceptable salt thereof in an amount of 60 mg once daily, and wherein said treatment achieves statistically significant least square mean difference in mean monthly migraine days in said patients from baseline to week 12 of said treatment as compared to placebo. In embodiments, treatment with atogepant achieves a least square mean difference in mean monthly migraine days from baseline to week 12 of said treatment of at least about −1, or at least about −1.2, or at least about −1.4, or at least about −1.6, or at least about −1.8 compared to placebo. In embodiments, treatment with atogepant achieves a least square mean difference in mean monthly migraine days from baseline to week 12 of said treatment of about −1.8 as compared to placebo. In embodiments, treatment with atogepant achieves a statistically significant improvement from baseline in migraine days within the first week of treatment, or within the first two weeks of treatment, or within the first three weeks of treatment, or within the first four weeks of treatment. In embodiments, treatment with atogepant achieves an improvement from baseline as early as the first full day after initiating treatment with atogepant.

In embodiments, the present disclosure provides a method for the preventive treatment of migraine, wherein the migraine is chronic migraine, comprising administering atogepant or a pharmaceutically acceptable salt thereof in an amount of 30 mg BID to a patient or patients in need thereof, wherein treatment with atogepant results in a ≥30% reduction in mean monthly migraine days (MMDs). In embodiments, a statistically significantly greater number of patients treated with atogepant 30 mg BID achieve ≥30% reduction in mean monthly migraine days across a 12-week treatment period as compared to placebo. In embodiments, at least about 50% of patients treated with atogepant, or at least about 55% of patients treated with atogepant, or at least about 60% of patients treated with atogepant, or at least about 61% of patients treated with atogepant, or at least about 62% of patients treated with atogepant, achieve a ≥30% reduction in MMDs across a 12-week treatment period.

In embodiments, the present disclosure provides a method for the preventive treatment of migraine, wherein the migraine is chronic migraine, comprising administering atogepant or a pharmaceutically acceptable salt thereof in an amount of 30 mg BID to a patient or patients in need thereof, wherein treatment with atogepant results in a ≥50% reduction in mean monthly migraine days. In embodiments, a statistically significantly greater number of patients treated with atogepant 30 mg BID achieve ≥50% reduction in mean monthly migraine days across a 12-week treatment period as compared to placebo. In embodiments, at least about 30% of patients treated with atogepant, or at least about 35%, or at least about 37%, or at least about 40%, or at least about 41%, or at least about 42% of patients treated with atogepant achieve a ≥50% reduction in MMDs across a 12-week treatment period.

In embodiments, the present disclosure provides a method for the preventive treatment of migraine, wherein the migraine is chronic migraine, comprising administering atogepant or a pharmaceutically acceptable salt thereof in an amount of 30 mg BID to a patient or patients in need thereof, wherein treatment with atogepant results in a ≥75% reduction in mean monthly migraine days. In embodiments, a statistically significantly greater number of patients achieve a ≥75% reduction in mean monthly migraine days as compared to placebo across a 12-week treatment period. In embodiments, at least about 10%, or at least about 15%, or at least about 16%, or at least about 17%, or at least about 18%, or at least about 19%, or at least about 20%, or at least about 21% of patients treated with atogepant achieve at least a ≥75% reduction in MMDs across a 12-week treatment period.

In embodiments, the present disclosure provides a method for the preventive treatment of migraine, wherein the migraine is chronic migraine, the method comprising administering atogepant or a pharmaceutically acceptable salt thereof in an amount of 60 mg QD to a patient or patients in need thereof, wherein treatment with atogepant results in a ≥30% reduction in mean monthly migraine days. In embodiments, a statistically significantly greater number of atogepant-treated patients achieves a ≥30% reduction in mean monthly migraine days as compared to placebo. In embodiments, at least about 50%, or at least about 55%, or at least about 56%, or at least about 57%, or at least about 58%, or at least about 59% of patients treated with atogepant achieve ≥30% reduction in mean monthly migraine days across a 12-week treatment period.

In embodiments, the present disclosure provides a method for the preventive treatment of migraine, wherein the migraine is chronic migraine, the method comprising administering atogepant or a pharmaceutically acceptable salt thereof in an amount of 60 mg QD to a patient or patients in need thereof, wherein treatment with atogepant achieves a ≥50% reduction in mean monthly migraine days. In embodiments, a statistically significantly greater number of atogepant-treated patients achieves a ≥50% reduction in mean monthly migraine days as compared to placebo. In embodiments, at least about 30%, or at least about 35%, or at least about 36%, or at least about 37%, or at least about 38%, or at least about 39%, or at least about 40%, or at least about 41% of patients treated with atogepant achieve ≥50% reduction in mean monthly migraine days across a 12-week treatment period.

In embodiments, the present disclosure provides a method for the preventive treatment of migraine, wherein the migraine is chronic migraine, the method comprising administering atogepant or a pharmaceutically acceptable salt thereof in an amount of 60 mg QD to a patient or patients in need thereof, wherein treatment with atogepant achieves a ≥75% reduction in mean monthly migraine days. In embodiments, a statistically significantly greater number of atogepant-treated patients achieve a ≥75% reduction in mean monthly migraine days as compared to placebo. In embodiments, at least about 10%, or at least about 15%, or at least about 16%, or at least about 17%, or at least about 18% of patients treated with atogepant achieve ≥75% reduction in mean monthly migraine days across a 12-week treatment period.

In embodiments, the present disclosure provides a method for the preventive treatment of migraine, such as the preventive treatment of chronic migraine, the method comprising administering atogepant in an amount of 60 mg once daily (QD) or 30 mg twice daily (BID) to a patient or patients in need thereof, wherein treatment with atogepant achieves a reduction from baseline in mean monthly headache days. In embodiments, treatment with atogepant 30 mg BID or 60 mg QD achieves a reduction in mean monthly headache days of at least about 6 days, or at least about 6.5 days, or at least about 6.7 days, or at least about 6.9 days, or at least about 7 days, or at least about 7.2 days, or at least about 7.3 days, or at least about 7.4 days.

In embodiments, the present disclosure provides a method for the preventive treatment of migraine in patients in need thereof, wherein the migraine is chronic migraine, comprising administering to each said patient atogepant or a pharmaceutically acceptable salt thereof in an amount of 60 mg once daily or 30 mg twice daily, and wherein said treatment achieves statistically significant least square mean difference in monthly headache days in said patients from baseline to week 12 of said treatment compared to placebo. In embodiments, treatment with atogepant achieves a least square mean difference in mean monthly headache days from baseline to week 12 of said treatment of at least about −1, or at least about −1.2, or at least about −1.4, or at least about −1.5, or at least about −1.6, or at least about −1.7, or at least about −1.8, or at least about −1.9, or at least about −2, or at least about −2.1, or at least about −2.2, or at least about −2.3 as compared to placebo.

In embodiments, the present disclosure provides a method for the preventive treatment of migraine, wherein the migraine is chronic migraine, the method comprising administering atogepant in an amount of 30 mg twice daily to a patient or patients in need thereof, and wherein treatment with atogepant achieves a reduction from baseline in mean monthly headache days. In embodiments, treatment with atogepant 30 mg twice daily achieves a reduction in mean monthly headache days at least about 6 days, or at least about 6.5 days, or at least about 6.7 days, or at least about 6.9 days, or at least about 7 days, or at least about 7.2 days, or at least about 7.3 days, or at least about 7.4 days. In embodiments, treatment with atogepant 30 mg BID achieves a reduction from baseline in mean monthly headache days of at least about 7.3 days across a 12-week treatment period. In embodiments, treatment with atogepant 30 mg BID achieves a reduction from baseline in mean monthly headache days of at least about 7.4 days across a 12-week treatment period.

In embodiments, the present disclosure provides a method for the preventive treatment of migraine in patients in need thereof, wherein the migraine is chronic migraine, comprising administering to said patients atogepant or a pharmaceutically acceptable salt thereof in an amount of 30 mg twice daily, and wherein said treatment achieves statistically significant least square mean difference in monthly headache days in said patients from baseline to week 12 of said treatment compared to placebo. In embodiments, treatment with atogepant 30 mg BID achieves a least square mean difference in mean monthly headache days from baseline to week 12 of at least about −1, or at least about −1.2, or at least about −1.4, or at least about −1.5, or at least about −1.6, or at least about −1.7, or at least about −1.8, or at least about −1.9, or at least about −2, or at least about −2.1, or at least about −2.2, or at least about −2.3 as compared to placebo. In embodiments, treatment with atogepant 30 mg twice daily achieves a least square mean difference in mean monthly headache days from baseline to week 12 of about −2.1 as compared to placebo. In embodiments, treatment with atogepant 30 mg twice daily achieves a least square mean difference in mean monthly headache days from baseline to week 12 of about −2.3 as compared to placebo.

In embodiments, the present disclosure provides a method for the preventive treatment of migraine, wherein the migraine is chronic migraine, the method comprising administering to a patient or patients in need thereof atogepant in an amount of 60 mg once daily, wherein treatment with atogepant achieves a reduction from baseline in mean monthly headache days. In embodiments, treatment with atogepant 60 mg once daily results in a reduction in mean monthly headache days of at least about 6 days, or at least about 6.5 days, or at least about 6.7 days, or at least about 6.9 days, or at least about 7 days. In embodiments, treatment with atogepant 60 mg achieves a reduction from baseline in mean monthly headache days of at least about 6.9 days across a 12-week treatment period. In embodiments, treatment with atogepant 60 mg achieves a reduction from baseline in mean monthly headache days of at least about 7 days across a 12-week treatment period.

In embodiments, the present disclosure provides a method for the preventive treatment of migraine in patients in need thereof, wherein the migraine is chronic migraine, comprising administering to said patient atogepant or a pharmaceutically acceptable salt thereof in an amount of 60 mg once daily, and wherein said treatment achieves statistically significant least square mean difference in monthly headache days from baseline to week 12 of said treatment compared to placebo. In embodiments, treatment with atogepant 60 mg once daily achieves a least square mean difference in mean monthly headache days from baseline to week 12 of at least about −1, or at least about −1.2, or at least about −1.4, or at least about −1.5, or at least about −1.6, or at least about −1.7, or at least about −1.8 compared to placebo. In embodiments, treatment with atogepant 60 mg once daily achieves a least square mean difference in mean monthly headache days from baseline to week 12 of about −1.7 compared to placebo. In embodiments, treatment with atogepant 60 mg once daily achieves a least square mean difference in mean monthly headache days from baseline to week 12 of about −1.8 compared to placebo.

In embodiments, treatment with atogepant 30 mg BID or atogepant 60 mg QD achieves a reduction from baseline in acute medication use days. In embodiments, the present disclosure provides a method for the preventive treatment of chronic migraine, the method comprising administering to a patient or patients in need thereof atogepant in an amount of 30 mg twice daily or 60 mg once daily, wherein treatment with atogepant achieves a change from baseline in mean monthly acute medication use days. In embodiments, acute medication use days refer to days on which a patient takes a medication to treat an acute migraine. Medications for the acute treatment of migraine include, for example, triptans, ergots, opioids, analgesics (including acetaminophen), NSAIDs (including aspirin), and antiemetics. In embodiments, treatment with atogepant 30 mg BID or 60 mg QD achieves a reduction from baseline in mean monthly acute medication use days of at least about 5 days, or at least about 5.5 days, or at least about 6 days, or at least about 6.1 days, or at least about 6.2 days, or at least about 6.3 days, or at least about 6.4 days, or at least about 6.5 days, or at least about 6.6 days, or at least about 6.7 days.

In embodiments, the present disclosure provides a method for the preventive treatment of migraine in patients in need thereof, wherein the migraine is chronic migraine, comprising administering to each said patient atogepant or a pharmaceutically acceptable salt thereof in an amount of 60 mg once daily or 30 mg twice daily, and wherein said treatment achieves statistically significant least square mean difference in monthly acute medication use days in said patients from baseline to week 12 of said treatment compared to placebo. In embodiments, treatment with atogepant 30 mg BID or atogepant 60 mg QD achieves a least square mean difference in mean monthly acute medication use days from baseline to week 12 of at least about −1, or at least about −1.5, or at least −2, or at least −2.1, or at least −2.2, or at least −2.3, or at least −2.4, or at least −2.5, or at least −2.6, as compared to placebo.

In embodiments, the present disclosure provides a method for the preventive treatment of migraine, wherein the migraine is chronic migraine, the method comprising administering to a patient or patients in need thereof atogepant in an amount of 30 mg twice daily, wherein treatment with atogepant achieves a reduction from baseline in mean monthly acute medication use days. In embodiments, treatment with atogepant 30 mg twice daily achieves a reduction from baseline in mean monthly acute medication use days of at least about 5 days, or at least about 5.5 days, or at least about 6 days, or at least about 6.1 days, or at least about 6.2 days, or at least about 6.3 days, or at least about 6.4 days, or at least about 6.5 days, or at least about 6.6 days, or at least about 6.7 days. In embodiments, treatment with atogepant 30 mg BID achieves a reduction from baseline in mean monthly acute medication use days of at least about 6.6 days. In embodiments, treatment with atogepant 30 mg BID achieves a reduction from baseline in mean monthly acute medication use days of at least about 6.7 days.

In embodiments, the present disclosure provides a method for the preventive treatment of migraine in patients in need thereof, wherein the migraine is chronic migraine, comprising administering to said patients atogepant or a pharmaceutically acceptable salt thereof in an amount of 30 mg twice daily, and wherein said treatment achieves statistically significant least square mean difference in monthly acute medication use days in said patients from baseline to week 12 of said treatment as compared to placebo. In embodiments, treatment with atogepant 30 mg BID achieves a least square mean difference in mean monthly acute medication use days from baseline to week 12 of at least about −1, or at least about −1.5, or at least about −2, or at least about −2.1, or at least about −2.2, or at least about −2.3, or at least about −2.4, or at least about −2.5, or at least about −2.6, compared to placebo. In embodiments, treatment with atogepant 30 mg BID achieves a least square mean difference in mean monthly acute medication use days from baseline to week 12 of at least about −2.5 as compared to placebo. In embodiments, treatment with atogepant 30 mg BID achieves a least square mean difference in mean monthly acute medication use days from baseline to week 12 of at least about −2.6 as compared to placebo.

In embodiments, the present disclosure provides a method for the preventive treatment of migraine, wherein the migraine is chronic migraine, the method comprising administering to a patient or patients in need thereof atogepant in an amount of 60 mg once daily, wherein treatment with atogepant achieves a reduction from baseline in mean monthly acute medication use days. In embodiments, treatment with atogepant 60 mg once daily results in a reduction in mean acute medication use days of at least about 5 days, or at least about 5.5 days, or at least about 6 days, or at least about 6.1 days, or at least about 6.2 days. In embodiments, treatment with atogepant 60 mg once daily achieves a reduction from baseline in mean acute medication use days of at least about 6.2 days across a 12-week treatment period.

In embodiments, the present disclosure provides a method for the preventive treatment of migraine in patients in need thereof, wherein the migraine is chronic migraine, comprising administering to said patients atogepant or a pharmaceutically acceptable salt thereof in an amount of 60 mg once daily, and wherein said treatment achieves statistically significant least square mean difference in monthly acute medication use days in said patients from baseline to week 12 of said treatment compared to placebo. In embodiments, treatment with atogepant 60 mg QD achieves a least square mean difference in mean monthly acute medication use days from baseline to week 12 of at least about −1, or at least about −1.5, or at least about −2, or at least about −2.1, compared to placebo. In embodiments, treatment with atogepant 60 mg once daily achieves a least square mean difference in mean monthly acute medication use days from baseline to week 12 of at least about −2.1 compared to placebo.

In embodiments, treatment with atogepant 30 mg BID or 60 mg QD achieves a reduction from baseline in the 3-month average of monthly migraine days. In embodiments, the present disclosure provides a method for the preventive treatment of migraine, wherein the migraine is chronic migraine, the method comprising administering to a patient or patients in need thereof atogepant in an amount of 30 mg twice daily or 60 mg once daily, wherein treatment with atogepant achieves a reduction from baseline in the 3-month average of monthly migraine days. In embodiments, treatment with atogepant 30 mg twice daily or 60 mg once daily achieves a statistically significant change from baseline in the proportion of patients with ≥50% reduction in 3-month average of monthly migraine days.

In embodiments of the present disclosure, treatment with atogepant results in an improvement (i.e., increase) from baseline in a MSQ v2.1 Role Function-Restrictive Domain Score. In embodiments, the present disclosure provides a method for the preventive treatment of migraine, wherein the migraine is chronic migraine, the method comprising administering to a patient or patients in need thereof atogepant in an amount of 30 mg twice daily or 60 mg once daily, wherein treatment with atogepant achieves an increase from baseline in a MSQ v2.1 Role Function-Restrictive Domain Score. In embodiments, treatment with atogepant 30 mg twice daily or 60 mg once daily achieves a statistically significant improvement from baseline in a MSQ v2.1 Role Function-Restrictive Domain Score.

The MSQ v2.1 is a 14-item questionnaire designed to measure health-related quality of life impairments attributed to migraine in the past 4 weeks. It is divided into three domains: Role Function Restrictive assesses how migraines limit one's daily social and work-related activities; Role Function Preventive assesses how migraines prevent these activities; and the Emotional Function domain assesses the emotions associated with migraine. Participants respond to items using a 6-point scale ranging from “none of the time” to “all of the time.” Raw dimension scores are computed as a sum of item responses and rescaled to a 0 to 100 scale, where higher scores indicate better quality of life.

In embodiments, the present disclosure provides a method for the preventive treatment of migraine, wherein the migraine is chronic migraine, the method comprising administering to a patient or patients in need thereof atogepant in an amount of 30 mg twice daily or 60 mg once daily, wherein treatment with atogepant achieves an increase from baseline in a MSQ v2.1 Role Function-Restrictive Domain Score of at least about 20 points, or at least about 21 points, or at least about 22 points, or at least about 23 points, or at least about 24 points, or at least about 25 points.

In embodiments, the present disclosure provides a method for the preventive treatment of migraine in patients in need thereof, wherein the migraine is chronic migraine, comprising administering to each said patient atogepant or a pharmaceutically acceptable salt thereof in an amount of 60 mg once daily or 30 mg twice daily, and wherein said treatment achieves statistically significant least square mean difference in MSQ v2.1 Role Function-Restrictive Domain Score in said patients from baseline to week 12 of said treatment compared to placebo. In embodiments, treatment with atogepant 30 mg BID or atogepant 60 mg QD achieves a least square mean difference in MSQ v2.1 Role Function-Restrictive Domain Score of at least about 4, or at least about 4.5, or at least about 5, or at least about 5.5, or at least about 5.7, or at least about 6, or at least about 6.5, or at least about 7, or at least about 7.4, or at least about 7.5, or at least about 7.6, or at least about 7.7, or at least about 7.8, or at least about 7.9, as compared to placebo.

In embodiments, the present disclosure provides a method for the preventive treatment of migraine, wherein the migraine is chronic migraine, the method comprising administering to a patient or patients in need thereof atogepant in an amount of 30 mg twice daily, wherein treatment with atogepant achieves an increase from baseline in MSQ v2.1 Role Function-Restrictive Domain Score. In embodiments, treatment with atogepant 30 mg twice daily achieves an increase from baseline in MSQ v2.1 Role Function-Restrictive Domain Score of at least about 20 points, or at least about 21 points, or at least about 22 points, or at least about 23 points, or at least about 24 points, or at least about 25 points. In embodiments, in a 12-week treatment period, treatment with atogepant 30 mg twice daily achieves an increase from baseline in MSQ v2.1 Role Function-Restrictive Domain Score of at least about 24.7 at week 12. In embodiments, in a 12-week treatment period, treatment with atogepant 30 mg twice daily achieves an increase from baseline in MSQ v2.1 Role Function-Restrictive Domain Score of at least about 25 at week 12.

In embodiments, the present disclosure provides a method for the preventive treatment of migraine in patients in need thereof, wherein the migraine is chronic migraine, comprising administering to said patients atogepant or a pharmaceutically acceptable salt thereof in an amount of 30 mg twice daily, and wherein said treatment achieves statistically significant least square mean difference in MSQ v2.1 Role Function-Restrictive Domain Score in said patients from baseline to week 12 of said treatment as compared to placebo. In embodiments, treatment with atogepant 30 mg BID achieves a least square mean difference in MSQ v2.1 Role Function-Restrictive Domain Score from baseline to week 12 of at least about 4, or at least about 4.5, or at least about 5, or at least about 5.5, or at least about 5.7, or at least about 6, or at least about 6.5, or at least about 7, or at least about 7.4, or at least about 7.5, or at least about 7.6, or at least about 7.7, or at least about 7.8, or at least about 7.9, as compared to placebo. In embodiments, treatment with atogepant 30 mg BID achieves a least square mean difference in MSQ v2.1 Role Function-Restrictive Domain Score from baseline to week 12 of at least about 7.4. In embodiments, treatment with atogepant 30 mg BID achieves a least square mean difference in MSQ v2.1 Role Function-Restrictive Domain Score from baseline to week 12 of at least about 7.9.

In embodiments, the present disclosure provides a method for the preventive treatment of migraine, wherein the migraine is chronic migraine, the method comprising administering to a patient or patients in need thereof atogepant in an amount of 60 mg once daily, wherein treatment with atogepant achieves an increase from baseline in MSQ v2.1 Role Function-Restrictive Domain Score. In embodiments, treatment with atogepant 60 mg once daily achieves an increase from baseline in MSQ v2.1 Role Function-Restrictive Domain Score of at least about 20, or at least about 21, or at least about 22, or at least about 23. In embodiments, in a 12 week treatment period, treatment with atogepant 60 mg QD achieves an increase from baseline in MSQ v2.1 Role Function-Restrictive Domain Score of at least about 22 at week 12.

In embodiments, the present disclosure provides a method for the preventive treatment of migraine in patients in need thereof, wherein the migraine is chronic migraine, comprising administering to said patients atogepant or a pharmaceutically acceptable salt thereof in an amount of 60 mg once daily, and wherein said treatment achieves statistically significant least square mean difference in MSQ v2.1 Role Function-Restrictive Domain Score in said patients from baseline to week 12 of said treatment compared to placebo. In embodiments, treatment with atogepant 60 mg QD achieves a least square mean difference in MSQ v2.1 Role Function-Restrictive Domain Score from baseline to week 12 of at least about 4, or at least about 4.5, or at least about 5, or at least about 5.5, or at least about 6, as compared to placebo. In embodiments, treatment with atogepant 60 mg QD achieves a least square mean difference in MSQ v2.1 Role Function-Restrictive Domain Score from baseline to week 12 of at least about 6.1 as compared to placebo.

In embodiments of the present disclosure, treatment with atogepant results in an improvement (decrease) from baseline in Mean Monthly Performance of Daily Activities Domain Score of the AIM-D scale. In embodiments, the present disclosure provides a method for the preventive treatment of migraine, wherein the migraine is chronic migraine, the method comprising administering to a patient or patients in need thereof atogepant or a pharmaceutically acceptable salt thereof in an amount of 30 mg twice daily or 60 mg once daily, wherein treatment with atogepant achieves an improvement from baseline in a Mean Monthly Performance of Daily Activities Domain Score of the AIM-D scale. In embodiments, treatment with atogepant results in an improvement from baseline in Mean Monthly Physical Impairment Domain Score of the AIM-D scale. In embodiments, the present disclosure provides a method for the preventive treatment of migraine, wherein the migraine is chronic migraine, the method comprising administering to a patient or patients in need thereof atogepant in an amount of 30 mg twice daily or 60 mg once daily, wherein treatment with atogepant achieves an improvement from baseline in a Mean Monthly Physical Impairment Domain Score of the AIM-D scale.

The AIM-D (Activity Impairment in Migraine — Diary) is an 11-function daily diary measure that assesses the impact of migraine. It is comprised of two domains that evaluate performance of daily activities (PDA: 7 items) and physical impairment (PI: 4 items). Participants are asked to rate the difficulty experienced in the past 24 hours with performance of daily activities (i.e., difficulty with household chores, errands, leisure activities at home, leisure of social activities outside the home, strenuous physical activities, concentrating and thinking clearly), and physical impairment (i.e., difficulty walking, moving body, bending forward, moving head) using the 6-point rating scale: “Not difficult at all,” “A little difficult,” “Somewhat difficult,” “Very Difficult,” “Extremely difficult,” and “I could not do it at all.” Three items include a response of “I did not . . . ” (i.e., “I did not have errands planned”). The AIM-D was developed as an electronic daily diary with the same set of questions administered in headache and non-headache versions. Raw domain scores are rescaled to a 0 to 100 scale, where higher scores indicate greater impact of migraine.

In embodiments, the present disclosure provides a method for the preventive treatment of migraine, wherein the migraine is chronic migraine, the method comprising administering to a patient or patients in need thereof atogepant or a pharmaceutically acceptable salt thereof in an amount of 30 mg twice daily or 60 mg once daily, wherein treatment with atogepant achieves a decrease from baseline in a Mean Monthly Performance of Daily Activities Score of the AIM-D of at least about 11 points, or at least about 11.5 points, or at least about 12 points, or at least about 12.5 points, or at least about 13 points, or at least about 13.5 points, or at least about 14 points, or at least about 14.1 points, or at least about 14.2 points.

In embodiments, the present disclosure provides a method for the preventive treatment of migraine in patients in need thereof, wherein the migraine is chronic migraine, comprising administering to each said patient atogepant or a pharmaceutically acceptable salt thereof in an amount of 60 mg once daily or 30 mg twice daily, and wherein said treatment achieves statistically significant least square mean difference in the Mean Monthly Performance of Daily Activities Score of the AIM-D in said patients from baseline to week 12 of said treatment compared to placebo. In embodiments, treatment with atogepant 30 mg BID or atogepant 60 mg QD achieves a least square mean difference in the Mean Monthly Performance of Daily Activities Score of the AIM-D from baseline to week 12 of at least about −1, or at least about −2, or at least about −2.5, or at least about −3, or at least about −3.3, or at least about −3.5, or at least about −3.8, or at least about −4, or at least about −4.5, or at least about −4.6, or at least about −4.7, or at least about −4.8 as compared to placebo.

In embodiments, the present disclosure provides a method for the preventive treatment of migraine, wherein the migraine is chronic migraine, the method comprising administering to a patient or patients in need thereof atogepant or a pharmaceutically acceptable salt thereof in an amount of 30 mg twice daily, wherein treatment with atogepant achieves a decrease from baseline in the Mean Monthly Performance of Daily Activities Score of the AIM-D. In embodiments, treatment with atogepant 30 mg twice daily achieves a decrease from baseline in the Mean Monthly Performance of Daily Activities Score of the AIM-D at least about 11 points, or at least about 11.5 points, or at least about 12 points, or at least about 12.5 points, or at least about 13 points, or at least about 13.5 points, or at least about 14 points, or at least about 14.1 points, or at least about 14.2 points. In embodiments, in a 12-week treatment period, treatment with atogepant 30 mg BID achieves a decrease from baseline in the Mean Monthly Performance of Daily Activities Score of the AIM-D of at least about 14.2 points.

In embodiments, the present disclosure provides a method for the preventive treatment of migraine in patients in need thereof, wherein the migraine is chronic migraine, comprising administering to said patients atogepant or a pharmaceutically acceptable salt thereof in an amount of 30 mg twice daily, and wherein said treatment achieves statistically significant least square mean difference in the Mean Monthly Performance of Daily Activities Score of the AIM-D in said patients from baseline to week 12 of said treatment compared to placebo. In embodiments, treatment with atogepant 30 mg BID achieves a least square mean difference in the Mean Monthly Performance of Daily Activities Score of the AIM-D from baseline to week 12 of said treatment of at least about −1, or at least about −2, or at least about −2.5, or at least about −3, or at least about −3.3, or at least about −3.5, or at least about −3.8, or at least about −4, or at least about −4.5, or at least about −4.6, or at least about −4.7, or at least about −4.8 compared to placebo. In embodiments, treatment with atogepant 30 mg BID achieves a least square mean difference in the Mean Monthly Performance of Daily Activities Score of the AIM-D from baseline to week 12 of said treatment of about −4.8 compared to placebo.

In embodiments, the present disclosure provides a method for the preventive treatment of migraine, wherein the migraine is chronic migraine, the method comprising administering to a patient or patients in need thereof atogepant or a pharmaceutically acceptable salt thereof in an amount of 60 mg once daily, wherein treatment with atogepant achieves a decrease from baseline in the Mean Monthly Performance of Daily Activities Score of the AIM-D. In embodiments, treatment with atogepant 60 mg once daily achieves a decrease from baseline in the Mean Monthly Performance of Daily Activities Score of the AIM-D of at least about 11 points, or at least about 11.5 points, or at least about 12 points, or at least about 12.5 points, or at least about 12.8 points. In embodiments, treatment with atogepant 60 mg QD achieves a decrease from baseline in the Mean Monthly Performance of Daily Activities Score of the AIM-D of at least about 12.8 points across a 12-week treatment period.

In embodiments, the present disclosure provides a method for the preventive treatment of migraine in patients in need thereof, wherein the migraine is chronic migraine, comprising administering to said patients atogepant or a pharmaceutically acceptable salt thereof in an amount of 60 mg once daily, and wherein said treatment achieves statistically significant least square mean difference in the Mean Monthly Performance of Daily Activities Score of the AIM-D in said patients from baseline to week 12 of said treatment compared to placebo. In embodiments, treatment with atogepant 60 mg QD achieves a least square mean difference in the Mean Monthly Performance of Daily Activities Score of the AIM-D from baseline to week 12 of said treatment of at least about −1, or at least about −2, or at least about −2.5, or at least about −3, or at least about −3.3 compared to placebo. In embodiments, treatment with atogepant 60 mg QD Achieves a least square mean difference in the Mean Monthly Performance of Daily Activities Score of the AIM-D from baseline to week 12 of said treatment of about −3.3 as compared to placebo.

In embodiments, the present disclosure provides a method for the preventive treatment of migraine, wherein the migraine is chronic migraine, the method comprising administering to a patient or patients in need thereof atogepant or a pharmaceutically acceptable salt thereof in an amount of 30 mg twice daily or 60 mg once daily, wherein treatment with atogepant achieves a decrease from baseline in a Mean Monthly Physical Impairment Domain Score of the AIM-D of at least about 9 points, or at least about 9.5 points, or at least about 10 points, or at least about 10.5 points, or at least about 11 points, or at least about 11.5 points, or at least about 12 points.

In embodiments, the present disclosure provides a method for the preventive treatment of migraine in patients in need thereof, wherein the migraine is chronic migraine, comprising administering to each said patient atogepant or a pharmaceutically acceptable salt thereof in an amount of 60 mg once daily or 30 mg twice daily, and wherein said treatment achieves statistically significant least square mean difference in the Mean Monthly Physical Impairment Domain Score of the AIM-D in said patients from baseline to week 12 of said treatment compared to placebo. In embodiments, treatment with atogepant 30 mg BID or atogepant 60 mg QD achieves a least square mean difference in the Mean Monthly Physical Impairment Domain Score of the AIM-D from baseline to week 12 of at least about −1, or at least about −1.5, or at least about −1.7, or at least about −2, or at least about −2.2, or at least about −2.5, or at least about −2.7, or at least about −3, or at least about −3.2, or at least about −3.5, or at least about −3.7, or at least about −4, or at least about −4.1, or at least about −4.2 as compared to placebo.

In embodiments, the present disclosure provides a method for the preventive treatment of migraine, wherein the migraine is chronic migraine, the method comprising administering to a patient or patients in need thereof atogepant or a pharmaceutically acceptable salt thereof in an amount of 30 mg twice daily, wherein treatment with atogepant achieves a decrease from baseline in the Mean Monthly Physical Impairment Domain Score of the AIM-D. In embodiments, treatment with atogepant 30 mg twice daily achieves a decrease from baseline in the Mean Monthly Physical Impairment Domain Score of the AIM-D of at least about 9 points, or at least about 10 points, or at least about 10.5 points, or at least about 11 points, or at least about 11.5 points, or at least about 12 points. In embodiments, treatment with atogepant 30 mg twice daily achieves a decrease from baseline in the Mean Monthly Physical Impairment Domain Score of the AIM-D of at least about 12.1 points.

In embodiments, the present disclosure provides a method for the preventive treatment of migraine in patients in need thereof, wherein the migraine is chronic migraine, comprising administering to said patients atogepant or a pharmaceutically acceptable salt thereof in an amount of 30 mg twice daily, and wherein said treatment achieves statistically significant least square mean difference in the Mean Monthly Physical Impairment Domain Score of the AIM-D in said patients from baseline to week 12 of said treatment compared to placebo. In embodiments, treatment with atogepant 30 mg BID achieves a least square mean difference in the Mean Monthly Physical Impairment Domain Score of the AIM-D from baseline to week 12 of at least about −1, or at least about −1.5, or at least about −1.7, or at least about −2, or at least about −2.2, or at least about −2.5, or at least about −2.7, or at least about −3, or at least about −3.2, or at least about −3.5, or at least about −3.7, or at least about −4, or at least about −4.1, or at least about −4.2 as compared to placebo. In embodiments, treatment with atogepant 30 mg BID achieves a least square mean difference in the Mean Monthly Physical Impairment Domain Score of the AIM-D from baseline to week 12 of about −4.2 as compared to placebo.

In embodiments, the present disclosure provides a method for the preventive treatment of migraine, wherein the migraine is chronic migraine, the method comprising administering to a patient or patients in need thereof atogepant or a pharmaceutically acceptable salt thereof in an amount of 60 mg once daily, wherein treatment with atogepant achieves a decrease from baseline in the Mean Monthly Physical Impairment Domain Score of the AIM-D. In embodiments, treatment with atogepant 60 mg once daily achieves a decrease from baseline in the Mean Monthly Physical Impairment Domain Score of the AIM-D of at least about 9 points, or at least about 9.5 points, or at least about 10 points, or at least about 10.5 points. In embodiments, treatment with atogepant 60 mg QD achieves a decrease from baseline to week 12 in the Mean Monthly Physical Impairment Domain Score of the AIM-D of about 10.6 points.

In embodiments, the present disclosure provides a method for the preventive treatment of migraine in patients in need thereof, wherein the migraine is chronic migraine, comprising administering to said patients atogepant or a pharmaceutically acceptable salt thereof in an amount of 60 mg once daily, and wherein said treatment achieves statistically significant least square mean difference in the Mean Monthly Physical Impairment Domain Score of the AIM-D in said patients from baseline to week 12 of said treatment compared to placebo. In embodiments, treatment with atogepant 60 mg QD achieves a least square mean difference in the Mean Monthly Physical Impairment Domain Score of the AIM-D from baseline to week 12 of at least about −1, or at least about −1.5, or at least about −1.7, or at least about −2, or at least about −2.2, or at least about −2.5, or at least about −2.7, as compared to placebo. In embodiments, treatment with atogepant 60 mg QD achieves a least square mean difference in the Mean Monthly Physical Impairment Domain Score of the AIM-D from baseline to week 12 of about −2.7

In embodiments of the present disclosure, treatment with atogepant results in an improvement (i.e., decrease) from baseline in the HIT-6 total score. In embodiments, the present disclosure provides a method for the preventive treatment of migraine, wherein the migraine is chronic migraine, the method comprising administering to a patient or patients in need thereof, atogepant in an amount of 30 mg twice daily or 60 mg once daily, wherein treatment with atogepant achieves an improvement (i.e., decrease) from baseline in a HIT-6 Total Score. In embodiments, treatment with atogepant achieves a statistically significant decrease from baseline in a HIT-6 Total Score.

The HIT-6 (Headache Impact Test) is a 6-question assessment used to measure the impact headaches have on a participant's ability to function on the job, at school, at home, and in social situations. It assesses the effect that headaches have on normal daily life and the participant's ability to function. Responses are based on frequency using a 5-point scale ranging from “never” to “always.” The HIT-6 total score, which ranges from 36 to 78, is the sum of the responses—each of which is assigned a score ranging from 6 points (never) to 13 points (always)—with higher scores indicating larger impact due to headache. Yang et al., “Validation of the Headache Impact Test (HIT-6™) across episodic and chronic migraine”, Cephalalgia, 2011 February; 31(3): 357−367.

In embodiments, the present disclosure provides a method for the preventive treatment of migraine, wherein the migraine is chronic migraine, the method comprising administering to a patient or patients in need thereof atogepant or a pharmaceutically acceptable salt thereof in an amount of 30 mg twice daily or 60 mg once daily, wherein treatment with atogepant achieves an reduction from baseline in the HIT-6 Total Score of at least about 6 points, or at least about 6.5 points, or at least about 7 points, or at least about 7.5 points, or at least about 7.8 points, or at least about 8 points, or at least about 8.2 points, or at least about 8.4 points.

In embodiments, the present disclosure provides a method for the preventive treatment of migraine in patients in need thereof, wherein the migraine is chronic migraine, comprising administering to each said patient atogepant or a pharmaceutically acceptable salt thereof in an amount of 60 mg once daily or 30 mg twice daily, and wherein said treatment achieves statistically significant least square mean difference in the HIT-6 Total Score in said patients from baseline to week 12 of said treatment compared to placebo. In embodiments, treatment with atogepant 30 mg BID or atogepant 60 mg QD achieves a least square mean difference in the HIT-6 Total Score of at least about −1, or at least about −1.5, or at least about −2, or at least about −2.5, or at least about −2.6, or at least about −2.8, or at least about −3, or at least about −3.1, or at least about −3.2, or at least about −3.3, as compared to placebo.

In embodiments, the present disclosure provides a method for the preventive treatment of migraine, wherein the migraine is chronic migraine, the method comprising administering to a patient or patients in need thereof atogepant or a pharmaceutically acceptable salt thereof in an amount of 30 mg twice daily, wherein treatment with atogepant achieves a decrease from baseline in HIT-6 total score. In embodiments, treatment with atogepant 30 mg twice daily achieves a decrease from baseline in HIT-6 total score of at least about 6 points, or at least about 6.5 points, or at least about 7 points, or at least about 7.5 points, or at least about 7.8 points, or at least about 8 points, or at least about 8.2 points, or at least about 8.4 points. In embodiments, treatment with atogepant 30 mg achieves a decrease from baseline to week 12 of said treatment in the HIT-6 total score of at least about 8.4 points.

In embodiments, the present disclosure provides a method for the preventive treatment of migraine in patients in need thereof, wherein the migraine is chronic migraine, comprising administering to said patients atogepant or a pharmaceutically acceptable salt thereof in an amount of 30 mg twice daily, and wherein said treatment achieves statistically significant least square mean difference in the HIT-6 Total Score in said patients from baseline to week 12 of said treatment compared to placebo. In embodiments, treatment with atogepant 30 mg BID achieves a least square mean difference in the HIT-6 Total Score of at least about −1, or at least about −1.5, or at least about −2, or at least about −2.5, or at least about −2.6, or at least about −2.8, or at least about −3, or at least about −3.1, or at least about −3.2, or at least about −3.3, as compared to placebo. In embodiments, treatment with atogepant 30 mg BID achieves a least square mean difference in the HIT-6 Total Score of about −3.3 at week 12 as compared to placebo.

In embodiments, the present disclosure provides a method for the preventive treatment of migraine, wherein the migraine is chronic migraine, the method comprising administering to a patient or patients in need thereof atogepant or a pharmaceutically acceptable salt thereof in an amount of 60 mg once daily, wherein treatment with atogepant achieves a decrease from baseline in HIT-6 total score. In embodiments, treatment with atogepant 60 mg once daily achieves a decrease from baseline in HIT-6 total score of at least about 6 points, or at least about 6.5 points, or at least about 7 points, or at least about 7.5 points, or at least about 7.8 points. In embodiments, treatment with atogepant 60 mg once daily achieves a decrease from baseline to week 12 of said treatment in HIT-6 total score of about 7.8 points.

In embodiments, the present disclosure provides a method for the preventive treatment of migraine in patients in need thereof, wherein the migraine is chronic migraine, comprising administering to said patients atogepant or a pharmaceutically acceptable salt thereof in an amount of 60 mg once daily, and wherein said treatment achieves statistically significant least square mean difference in the HIT-6 Total Score in said patients from baseline to week 12 of said treatment compared to placebo. In embodiments, treatment with atogepant 60 mg QD achieves a least square mean difference in the HIT-6 Total Score from baseline to week 12 of said treatment of at least about −1, or at least about −1.5, or at least about −2, or at least about −2.5, or at least about −2.6, as compared to placebo. In embodiments, treatment with atogepant 60 mg QD achieves a least square mean difference in the HIT-6 Total Score from baseline to week 12 of said treatment of about −2.6, as compared to placebo.

### EXAMPLES

### Example 1

A phase 3, multicenter, randomized, double-blind, placebo-controlled, parallel-group study was carried out to evaluate the efficacy, safety, and tolerability of atogepant for the prevention of chronic migraine. A total of 778 participants from 133 sites in North America, Europe, and East Asia were randomized to one of three treatment arms (placebo, Atogepant 30 mg BID, and Atogepant 60 mg QD) in a 1:1:1 ratio.

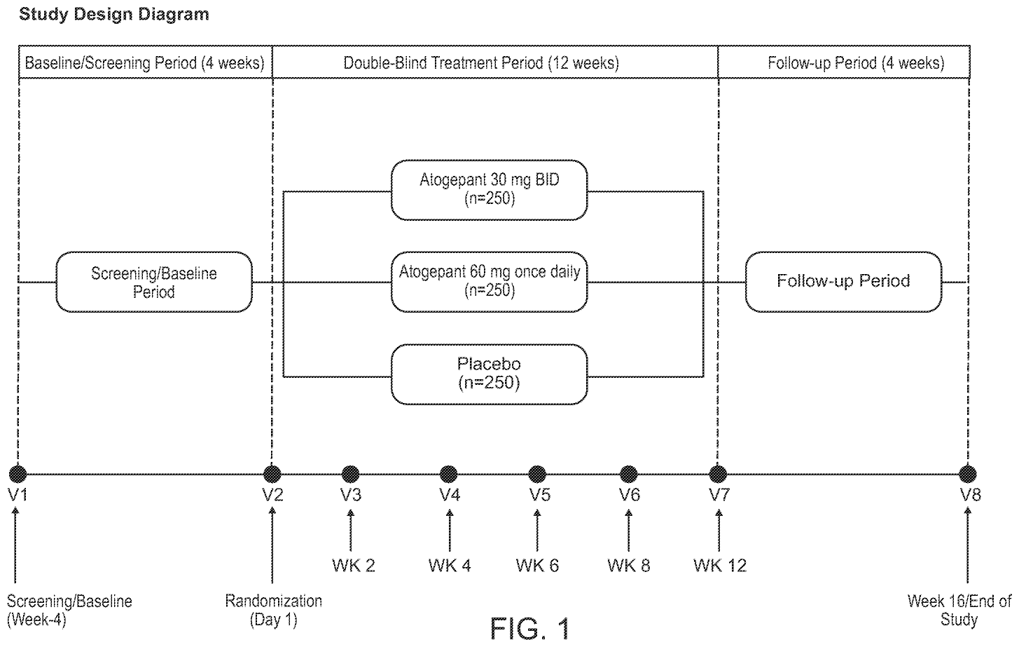

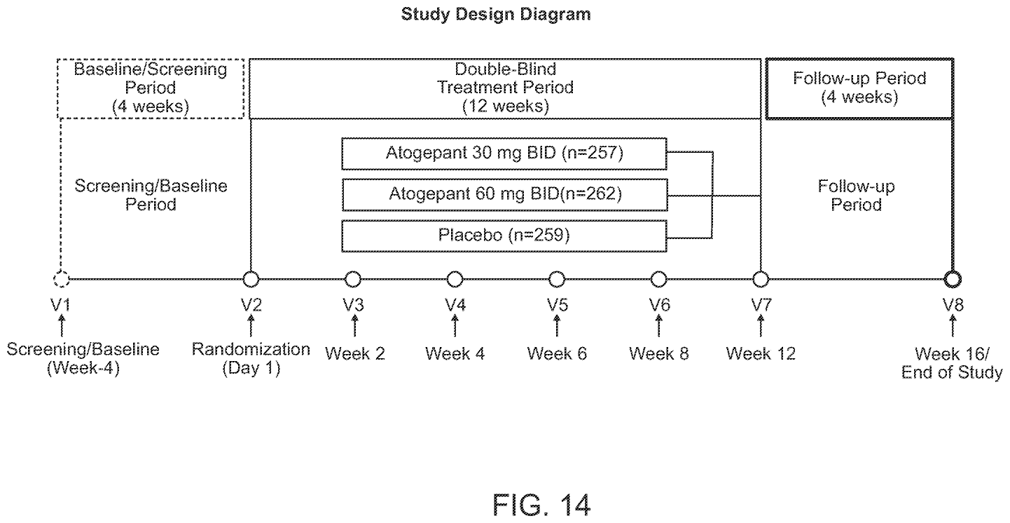

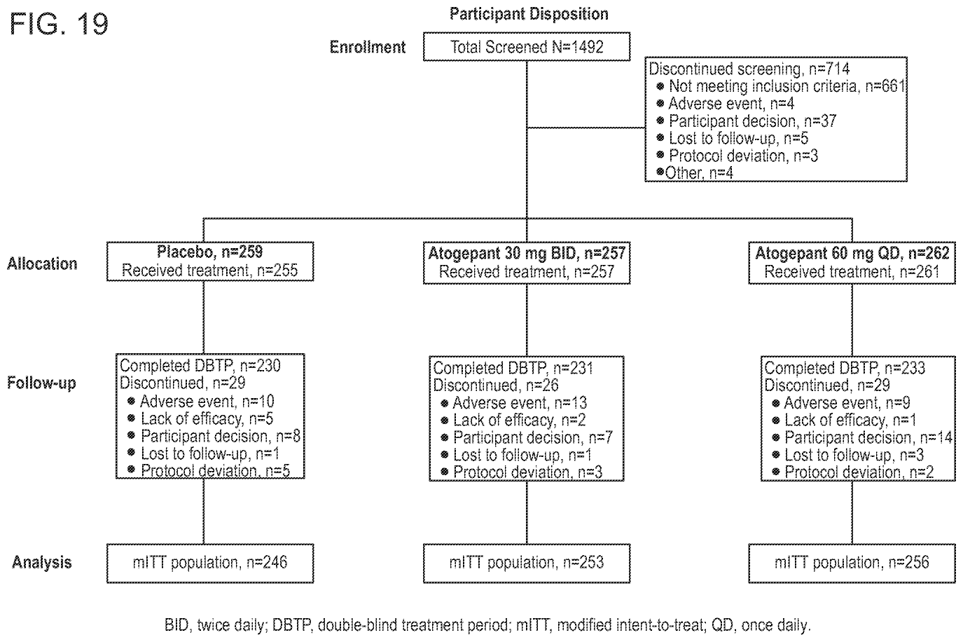

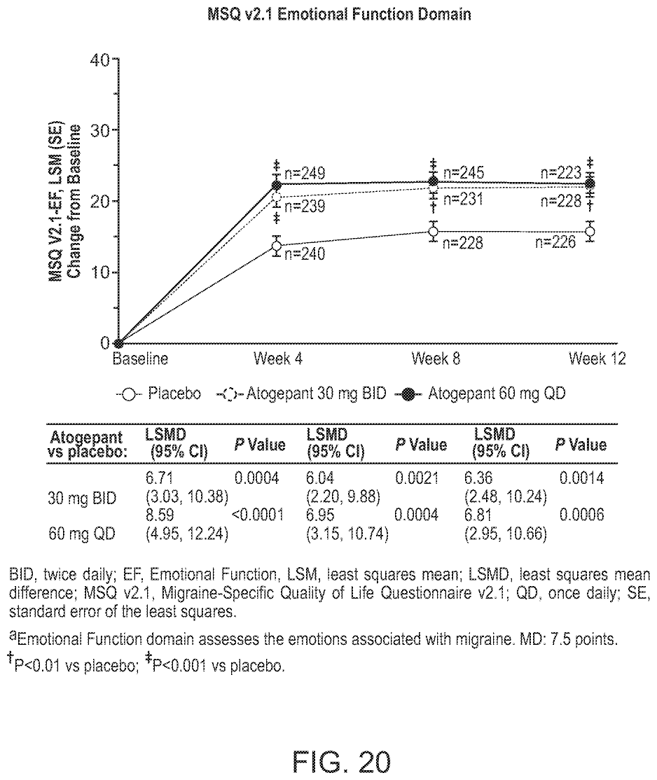

This study comprised a 4-week screening and baseline period, a 12-week double blind treatment period, and a follow-up period of 4 additional weeks. The total study duration was 20 weeks. Study design is summarized in FIG. 1 and FIG. 14. Participant disposition information and other relevant participant screening information may also be found in FIG. 19.

The primary end point was the change from baseline in the mean number of migraine days per month across the 12 week treatment period.

Secondary endpoints included change from baseline in mean monthly headache days across the 12 week treatment period; change from baseline in mean monthly acute medication use days across the 12-week treatment period; at least a 50% reduction in mean monthly migraine days across the 12-week treatment period; change from baseline in MSQ v2.1 Role Function-Restrictive domain score at week 12; change from baseline in mean AIM-D monthly functioning and activity impairment score across the 12-week treatment period; and change from baseline in HIT-6 total score at week 12.

Inclusion criteria included at least a 1-year history of chronic migraine consistent with diagnosis according to the International Classification of Headache Disorders, 3rd Edition (ICHD-3) and age of the participant at the time of migraine onset <50 years. Inclusion criteria further included confirmation of headache / migraine headache day frequency as follows: history of, on average, ≥15 headache days per month in the 3 months prior to Visit 1 in the opinion of the investigator AND >=15 headache days during the 4-week screening/baseline period per the electronic diary (eDiary) AND >=8 days during the 4-week screening/baseline period that qualify as being a migraine day per the eDiary. Participants were also required to be using a medically acceptable and effective method of birth control during the course of the entire study.

Exclusion criteria included a history of migraine, accompanied by diplopia or decreased level of consciousness, or retinal migraine; a current diagnosis of new persistent daily headache, trigeminal autonomic cephalgia (e.g., cluster headache), or painful cranial neuropathy; history of an inadequate response to >4 medications (2 of which have different mechanisms of action) prescribed for the prevention of migraine; and woman who is pregnant, planning to become pregnant during the course of the study, or currently lactating.

The trial included 778 randomized participants (259 to placebo, 257 to atogepant 30 mg BID, 262 to atogepant 60 mg QD) with at least a one-year history of chronic migraine. The safety population included 773 participants and the modified-intent-to-treat population included 755 participants. The subject population is summarized in table 1.

TABLE 1
Subject Population

| | | Atogepant | Atogepant | |
|---|---|---|---|---|
| Population | Placebo | 30 mg BID | 60 mg QD | Overall |
| Screened | | | | 1489 |
| Intent-to-Treat | 259 | 257 | 262 | 778 |
| Safety | 255 | 257 | 261 | 773 |
| Modified Intent-to-Treat | 246 | 253 | 256 | 755 |
| Off-Treatment | 249 | 254 | 257 | 760 |
| Hypothetical Estimand | | | | |

All Screened Participants includes those screened participants who signed informed consent form.
The Intent-to-Treat Population includes all randomized participants. Treatment group assigned as randomized.
The Modified Intent-to-Treat (mITT) population includes all randomized participants who received at least 1 dose of study intervention, had an evaluable baseline period of eDiary data, and had at least 1 evaluable post-baseline 4-week period (Weeks 1-4, 5-8, and 9-12) of eDiary data during the double blind treatment period. Treatment group assigned as randomized.
The analysis population for Off-treatment Hypothetical Estimand (OTHE) includes all randomized participants who received at least one dose of study treatment, had an evaluable baseline period of eDiary data and had at least one evaluable post-baseline 4-week period (weeks 1-4, 5-8, 9-12) of eDiary data during the double blind treatment period and follow-up period, regardless of whether on study treatment or off study treatment. Treatment groups assigned as randomized.

Randomization was stratified by region (North America 29.3%, Europe 35.3%, East Asia 35.3%), exposure to prior migraine prevention medication with proven efficacy (current use 10.7%, past use 70.1%, never used 19.3%), and number of failed medications (failed 0 or 1 or more with the same mechanism of action 42.8%, failed 2 or more with different mechanisms of action 37.9%), and acute headache medication overuse (Yes 65.3%, No 34.7%).

Baseline demographics of the safety population are provided in Table 2. The demographics were generally balanced among treatment groups.

TABLE 2
Baseline Demographics Safety Population

| | | | Atogepant | | Atogepant | | | |
|---|---|---|---|---|---|---|---|---|
| | Placebo | | 30 mg BID | | 60 mg QD | | Total | |
| Parameter | (N = 255) | | (N = 257) | | (N = 261) | | (N = 773) | |
| Age (years), Mean (SD) | 42.0 | (12.43) | 42.6 | (11.89) | 41.7 | (12.31) | 42.1 | (12.20) |
| Sex, n (%) | | | | | | | | |
| Male | 30 | (11.8) | 30 | (11.7) | 36 | (13.8) | 96 | (12.4) |
| Female | 225 | (88.2) | 227 | (88.3) | 225 | (86.2) | 677 | (87.6) |
| Race, n (%) | | | | | | | | |
| White | 151 | (59.2) | 151 | (58.8) | 157 | (60.2) | 459 | (59.4) |
| Black or African | 7 | (2.7) | 8 | (3.1) | 9 | (3.4) | 24 | (3.1) |
| American | | | | | | | | |
| Asian | 94 | (36.9) | 95 | (37.0) | 92 | (35.2) | 281 | (36.4) |
| American Indian Or | 1 | (0.4) | 1 | (0.4) | 1 | (0.4) | 3 | (0.4) |
| Alaska Native | | | | | | | | |
| Native Hawaiian Or | 1 | (0.4) | 0 | | 0 | | 1 | (0.1) |
| Other | | | | | | | | |
| Pacific Islander | | | | | | | | |
| Multiple | 1 | (0.4) | 2 | (0.8) | 2 | (0.8) | 5 | (0.6) |
| Ethnicity, n (%) | | | | | | | | |
| Hispanic | 13 | (5.1) | 12 | (4.7) | 6 | (2.3) | 31 | (4.0) |
| Non-Hispanic | 242 | (94.9) | 245 | (95.3) | 255 | (97.7) | 742 | (96.0) |
| Region | | | | | | | | |
| North America | 75 | (29.4) | 74 | (28.8) | 79 | (30.3) | 228 | (29.5) |
| Europe | 89 | (34.9) | 92 | (35.8) | 91 | (34.9) | 272 | (35.2) |
| East Asia | 91 | (35.7) | 91 | (35.4) | 91 | (34.9) | 273 | (35.3) |

Migraine and migraine treatment histories for the safety population are provided in Table 3 and Table 3a, respectively.

TABLE 3
Migraine History - Safety Population

| | | | Atogepant | | Atogepant | | | |
|---|---|---|---|---|---|---|---|---|
| | Placebo | | 30 mg BID | | 60 mg QD | | Overall | |
| Parameter | (N = 255) | | (N = 257) | | (N = 261) | | (N = 773) | |
| Migraine Diagnosis, n (%) | | | | | | | | |
| With Aura | 35 | (13.7) | 34 | (13.2) | 34 | (13.0) | 103 | (13.3) |
| Without Aura | 151 | (59.2) | 153 | (59.5) | 158 | (60.5) | 462 | (59.8) |
| Both | 69 | (27.1) | 70 | (27.2) | 69 | (26.4) | 208 | (26.9) |
| Migraine Duration in Years, | 20.7 | (12.32) | 22.2 | (12.31) | 21.2 | (11.91) | 21.4 | (12.18) |
| Mean (SD) | | | | | | | | |
| Past Use of Migraine Prevention, | | | | | | | | |
| n (%) | | | | | | | | |
| Yes | 212 | (83.1) | 215 | (83.7) | 214 | (82.0) | 641 | (82.9) |
| No | 43 | (16.9) | 42 | (16.3) | 47 | (18.0) | 132 | (17.1) |
| Average # Migraine Days | 15.7 | (5.71) | 15.9 | (5.48) | 16.6 | (6.47) | 16.0 | (5.91) |
| Per Month in Last 3 Months, | | | | | | | | |
| Mean (SD) | | | | | | | | |
| Average # Headache Days | 20.8 | (4.99) | 20.8 | (4.77) | 21.7 | (6.18) | 21.1 | (5.36) |

Per Month in Last 3 Months,
Mean (SD)

TABLE 3a
Migraine Treatment History - Safety Population

| Acute migraine treatment, n (%) | | | | | | | | |
|---|---|---|---|---|---|---|---|---|
| Yes | 250 | (98.0) | 252 | (98.1) | 258 | (98.9) | 760 | (98.3) |
| Triptan | 189 | (74.1) | 185 | (72.0) | 184 | (70.5) | 558 | (72.2) |
| Ergot or ergot combination | 1 | (0.4) | 6 | (2.3) | 10 | (3.8) | 17 | (2.2) |
| NSAID | 189 | (74.1) | 170 | (66.1) | 181 | (69.3) | 540 | (69.9) |
| Opioid or opioid combination | 9 | (3.5) | 11 | (4.3) | 8 | (3.1) | 28 | (3.6) |
| Antiemetic | 36 | (14.1) | 38 | (14.8) | 42 | (16.1) | 116 | (15.0) |
| Barbiturate | 0 | | 1 | (0.4) | 1 | (0.4) | 2 | (0.3) |
| Other | 84 | (32.9) | 79 | (30.7) | 87 | (33.3) | 250 | (32.3) |
| No | 5 | (2.0) | 5 | (1.9) | 3 | (1.1) | 13 | (1.7) |

A Subset of patients (11%) was allowed to use one concomitant migraine preventive medication (e.g., amitriptyline, propranolol, topiramate). Patients were allowed to use acute headache treatments (i.e., triptans, ergotamine derivatives, NSAIDs, acetaminophen, and opioids) as needed. Patients with acute medication overuse and medication overuse headache were also enrolled. The use of a concomitant medication that acts on the CGRP pathway was not permitted for either acute or preventive treatment of migraine. The study excluded patients with myocardial infarction, stroke, or transient ischemic attacks within six months prior to screening.

Primary and secondary efficacy endpoint analyses for the mITT population are presented in Table 4. The primary endpoint of statistically and clinically significant reduction from baseline in mean monthly migraine days compared to placebo was met for both the 60 mg once daily (QD) and 30 mg twice daily (BID) doses across the 12-week treatment period. The study also demonstrated that treatment with atogepant 60 mg QD and 30 mg BID resulted in statistically and clinically significant improvements in all secondary endpoints after adjustment for multiple comparisons.

TABLE 4
Summary of Primary and Secondary Efficacy Analyses
with Multiplicity Adjustment - mITT population

| | | Atogepant | Atogepant |
|---|---|---|---|
| | Placebo | 30 mg BID | 60 mg QD |
| Key Efficacy Endpoints | (N = 246) | (N = 253) | (N = 256) |
| P1: Change from baseline in mean monthly | | | |
| migraine days across the 12-week treatment | | | |
| period | | | |
| Baseline number of monthly migraine days, | 18.95 | 18.56 | 19.19 |
| Mean (SD) | (4.775) | (5.066) | (5.280) |
| LS Mean (SE) | −5.05 | −7.46 | −6.88 |
| | (0.411) | (0.405) | (0.406) |
| LSMD (95% CI), Atogepant vs. Placebo | | −2.41 | −1.82 |
| | | (−3.48, −1.33) | (−2.89, −0.75) |
| Nominal p-value from model | | <0.0001 | 0.0009 |
| Adjusted p-value | | <0.0001 | 0.0009 |
| S1: Change from baseline in mean monthly | | | |
| headache days across the 12-week treatment | | | |
| period | | | |
| Baseline number of monthly headache days, | 21.40 | 21.14 | 21.52 |
| Mean (SD) | (4.101) | (4.131) | (4.319) |
| LS Mean (SE) | −5.13 | −7.44 | −7.00 |
| | (0.405) | (0.399) | (0.401) |
| LSMD (95% CI), Atogepant vs. Placebo | | −2.32 | −1.87 |
| | | (−3.38, −1.26) | (−2.93, −0.81) |
| Nominal p-value from model | | <0.0001 | 0.0005 |
| Adjusted p-value | | <0.0001 | 0.0009 |
| S2: Change from baseline in mean monthly acute | | | |
| medication use days across the 12-week treatment | | | |
| period | | | |
| Baseline number of monthly acute medication use | 15.42 | 14.47 | 15.46 |
| days, Mean (SD) | (6.991) | (7.187) | (7.377) |
| LS Mean (SE) | −4.10 | −6.73 | −6.23 |
| | (0.392) | (0.389) | (0.386) |
| LSMD (95% CI), Atogepant vs. Placebo | | −2.63 | −2.13 |
| | | (−3.63, −1.63) | (−3.13, −1.13) |
| Nominal p-value from model | | <0.0001 | <0.0001 |
| Adjusted p-value | | <0.0001 | 0.0009 |
| S3: >=50% reduction in 3-month average of | | | |
| monthly migraine days | | | |
| Responders, n (%) | 64 | 108 | 105 |
| | (26.0) | (42.7) | (41.0) |
| Odds ratio (95% CI), Atogepant vs. Placebo | | 2.13 | 2.04 |
| | | (1.45, 3.14) | (1.38, 3.00) |
| Nominal p-value from model | | 0.0001 | 0.0003 |
| Adjusted p-value | | 0.0003 | 0.0009 |
| S4: Change from baseline in MSQ v2.1 role | | | |
| function restrictive domain score at week 12 | | | |
| Baseline MSQ v2.1 role function restrictive domain | 43.55 | 44.0 | 43.56 |
| score, Mean (SD) | (19.047) | (19.027) | (18.907) |
| LS Mean (SE) | 17.18 | 25.15 | 23.33 |
| | (1.381) | (1.373) | (1.365) |
| LSMD (95% CI), Atogepant vs. Placebo | | 7.96 | 6.15 |
| | | (4.30, 11.63) | (2.51, 9.79) |
| Nominal p-value from model | | <0.0001 | 0.0009 |
| Adjusted p-value | | 0.0003 | 0.0009 |
| S5: Change from baseline in mean monthly | | | |
| performance of daily activities domain score of | | | |
| the AIM-D across the 12-week treatment period | | | |
| Baseline monthly performance of daily activities | 29.50 | 29.28 | 31.18 |
| domain score, Mean (SD) | (13.733) | (15.062) | (16.470) |
| LS Mean (SE) | −9.44 | −14.29 | −12.82 |
| | (0.720) | (0.722) | (0.718) |
| LSMD (95% CI), Atogepant vs. Placebo | | −4.85 | −3.38 |
| | | (−6.75, −2.95) | (−5.27, −1.49) |
| Nominal p-value from model | | <0.0001 | 0.0005 |
| Adjusted p-value | | 0.0003 | 0.0009 |
| S6: Change from baseline in mean monthly | | | |
| physical impairment domain score of the AIM-D | | | |
| across the 12-week treatment period | | | |
| Baseline monthly physical impairment domain score, | 25.24 | 25.36 | 27.11 |
| Mean (SD) | (13.522) | (15.416) | (16.630) |
| LS Mean (SE) | −7.92 | −12.11 | −10.63 |
| | (0.667) | (0.668) | (0.665) |
| LSMD (95% CI), Atogepant vs. Placebo | | −4.19 | −2.71 |
| | | (−5.95, −2.43) | (−4.47, −0.96) |
| Nominal p-value from model | | <0.0001 | 0.0025 |
| Adjusted p-value | | 0.0003 | 0.0025 |

Of 1489 screened participants, 755 were included in the modified intent-to-treat population (atogepant 30 mg BID, n=253; atogepant 60 mg QD, n=256; placebo, n=246). Baseline mean (SE) number of MMDs were 18.6 (5.1) with atogepant 30 mg BID, 19.2 (5.3) with atogepant 60 mg QD, and 18.9 (4.8) with placebo. Changes from baseline in mean MMDs across 12 weeks were −7.5 (SE 0.4) days with atogepant 30 mg BID, −6.9 (SE 0.4) days with atogepant 60 mg QD, and −5.1 (SE 0.4) days with placebo. Least square mean differences from placebo were −2.4 days with atogepant 30 mg BID (95% CI −3.5 to −1.3; adjusted p<0.0001) and −1.8 days with atogepant 60 mg QD (95% CI −2.9 to −0.8; adjusted p=0.0009). Most common (≥5%) adverse events in the atogepant groups were constipation (30 mg BID: 28 [10.9%]; 60 mg QD: 26 [10%]; placebo: 8 [3.1%]) and nausea (30 mg BID: 20 [7.8%]; 60 mg QD: 25 [9.6%]; placebo: 9 [3.5%]). Across the 12 weeks, based on the mITT population, patients in the atogepant 60 mg QD and 30 mg BID treatment arms of the study experienced a decrease of 6.88 and 7.46 monthly migraine days, respectively, compared to patients in the placebo arm, who experienced a decrease of 5.05 monthly migraine days (60 mg QD vs. placebo, p=0.0009; 30 mg BID vs. placebo, p<0.0001, adjusted for multiple comparisons). Atogepant 30 mg BID and 60 mg QD demonstrated clinically relevant reductions in MMDs across 12 weeks. Both doses of atogepant were well tolerated, and results were consistent with the known safety profile of atogepant.

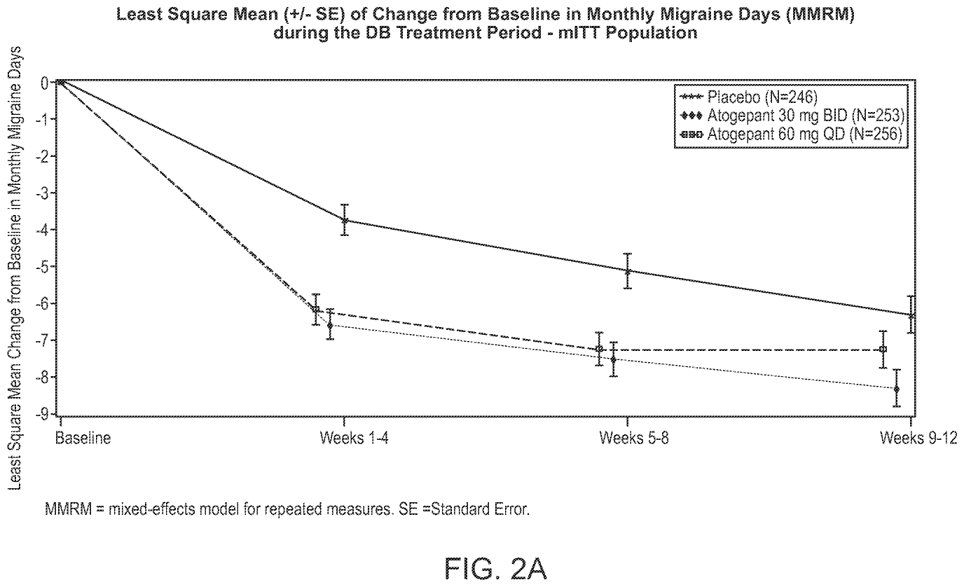

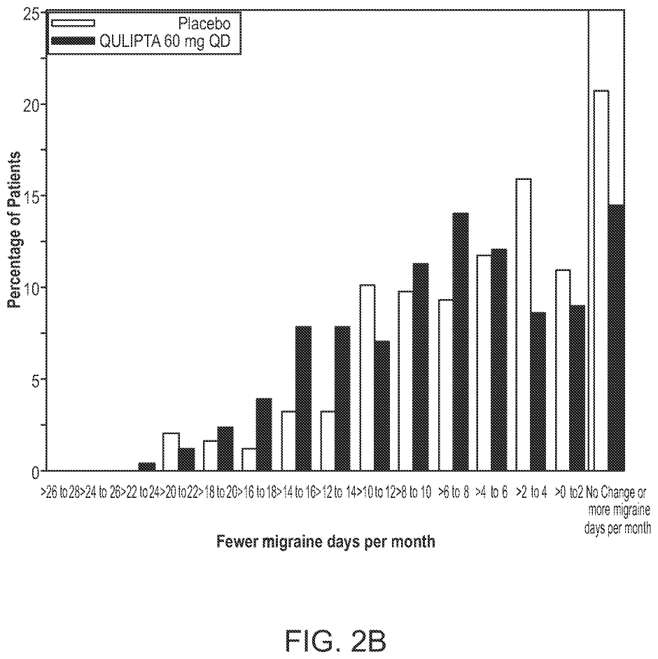

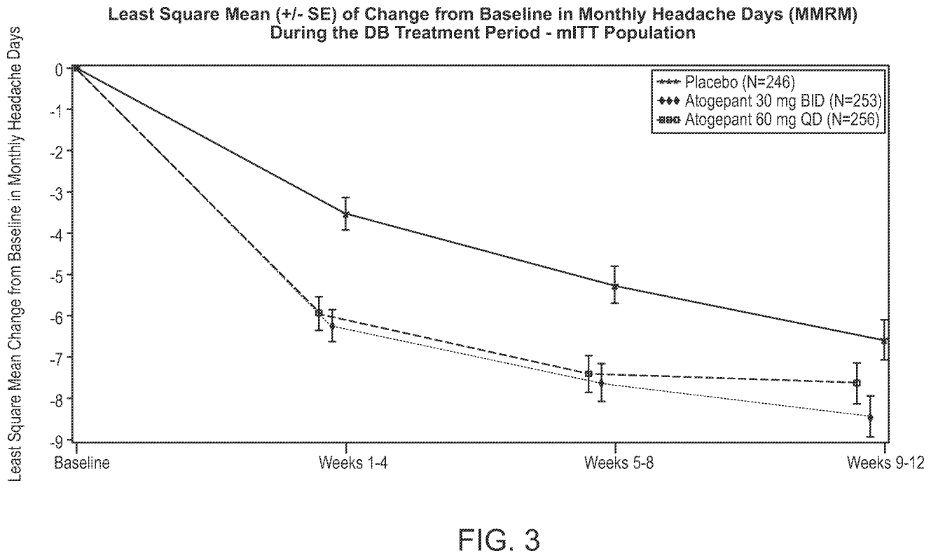

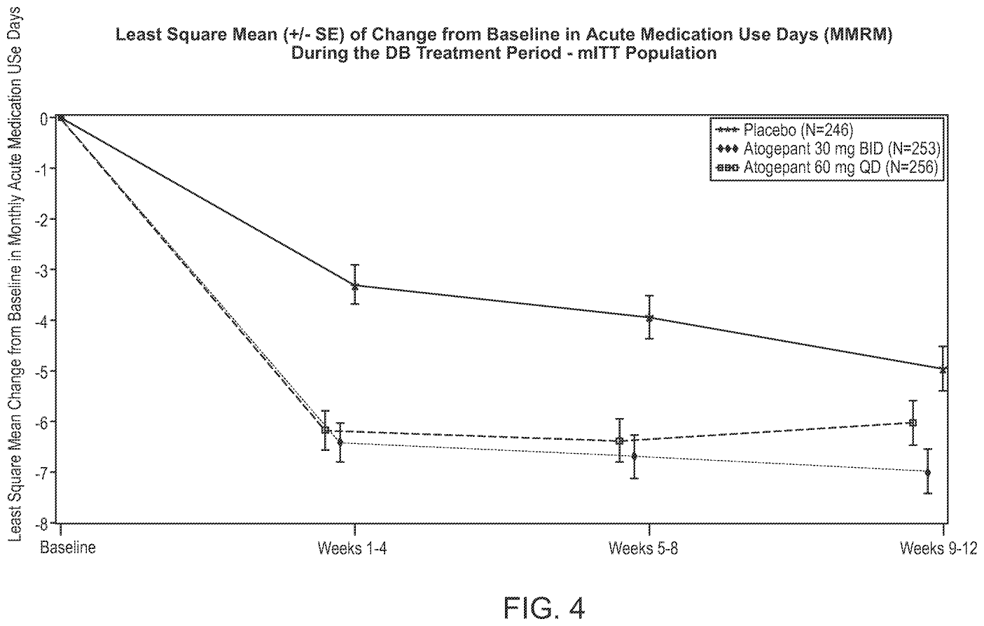

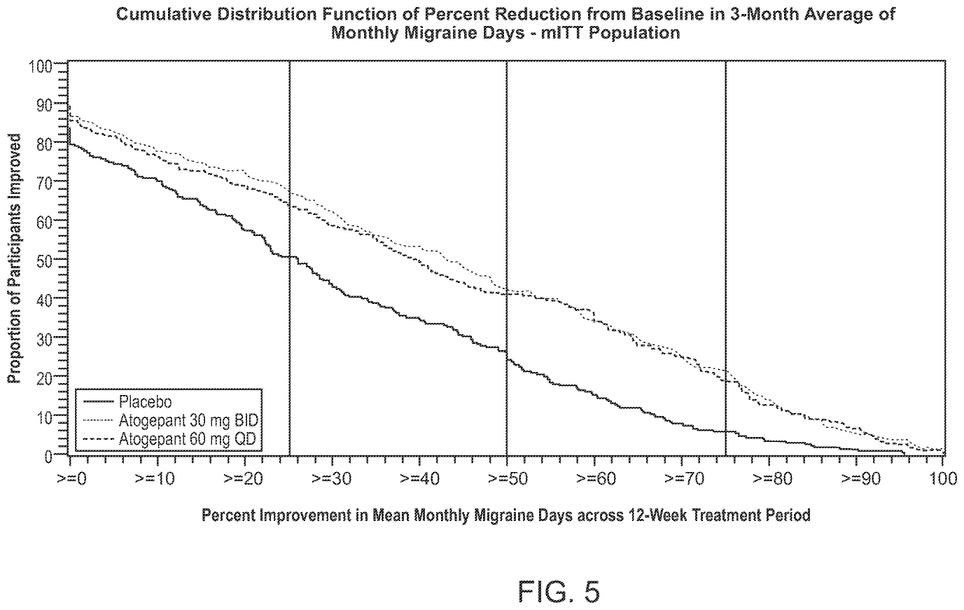

FIG. 2A shows the least square mean (+/−SE) of change from baseline in monthly migraine days (MMRM) during the double-blind treatment period in the mITT population. FIG. 3 shows the least square mean (+/−SE) of change from baseline in monthly headache days (MMRM) during the double blind treatment period for the mITT population. FIG. 4 shows the least square mean (+/−SE) of change from baseline in acute medication use days (MMRM) during the double blind treatment period for the mITT population. FIG. 5 shows the cumulative distribution function of percent reduction from baseline in 3-month average of monthly migraine days in the mITT population. As shown in Table 4 and FIGS. 2-5, the primary efficacy endpoints and key secondary endpoints demonstrated statistically significant improvement between each atogepant dose group and placebo.

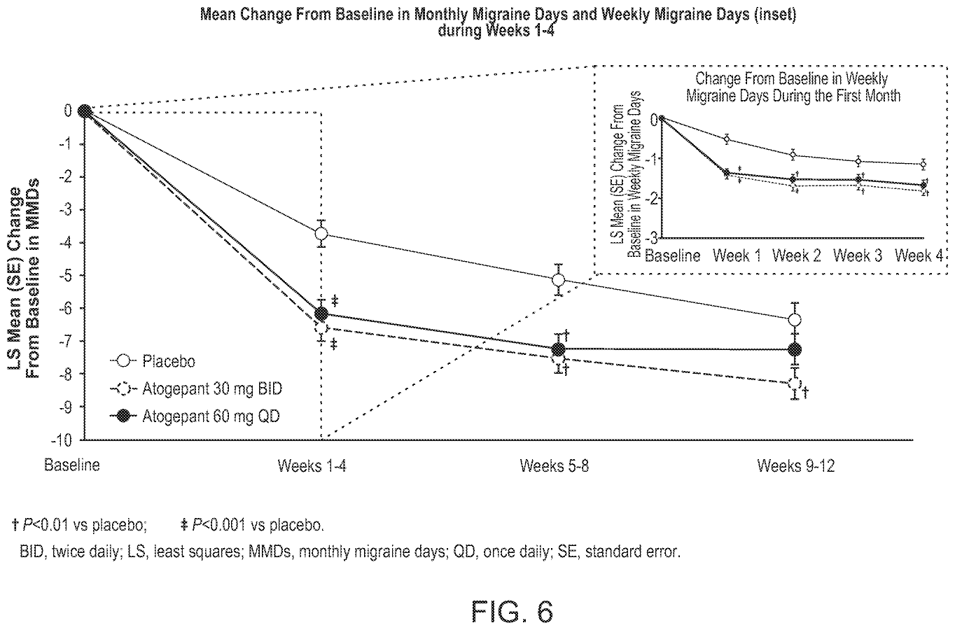

Outcomes for this analysis included change from baseline in mean MMDs during 4-week intervals, change in weekly migraine days during weeks 1-4, and the proportion of participants with a migraine on each day during the first 7 days of treatment. In adults with CM, atogepant demonstrated an early and sustained reduction in migraine days. Results showed a statistically significant effect of treatment during each week of the first 4-week treatment intervals, and as early as the first full day after study drug initiation. FIG. 6 shows mean changes from baseline in monthly migraine days for weeks 1-4, weeks 5-8, and weeks 9-12, and weekly migraine days during weeks 1-4 (inset). Baseline MMDs ranged from 18.6 to 19.2 across treatment groups. During weeks 1-4 of treatment, mean changes from baseline in MMDs were −6.6 for atogepant 30 mg BID, −6.2 for atogepant 60 mg QD, and −3.7 for placebo (P<0.001). During the following 4-week interval, this significantly greater MMD decrease in the atogepant treatment groups compared with placebo was maintained (P<0.001). Baseline weekly migraine days ranged from 4.6 to 4.8 across treatment groups. During each week of the first month of treatment, mean reduction in weekly migraine days were greater in both atogepant treatment groups compared with placebo (P≤0.009). During the 28-day baseline period, the daily rate of participants reporting a migraine day ranged from 66.3% to 68.4% across treatment groups. On the first full day after treatment initiation, atogepant-treated participants were significantly less likely to have a migraine than those who received placebo (P≤0.03).

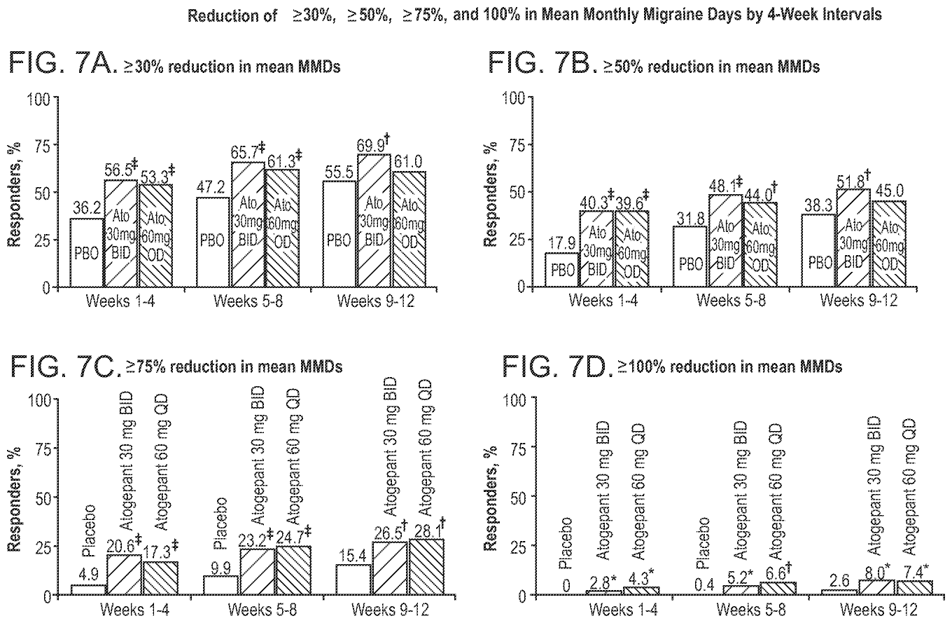

Atogepant treated participants (30 mg BID and 60 mg QD) were significantly more likely than placebo-treated participants, respectively, to experience a ≥30% (62.1% and 59.0% vs 43.1%; P<0.001), ≥50% (42.7% and 41.0% vs 26.0%; P<0.001), or ≥75% (21.3% and 18.8% vs 5.7%; P<0.001) reduction in mean MMDs across 12 weeks. During weeks 1-4 and 5-8, the proportion of participants experiencing a ≥30% or ≥50% reduction in mean MMDs was significantly greater for both atogepant doses vs placebo and during weeks 9-12 for atogepant 30 mg BID vs placebo. FIG. 7 shows the proportion of patients treated with atogepant 30 mg BID, atogepant 60 mg QD, and placebo who experienced a ≥30%, ≥50%, ≥75%, or 100% reduction in MMDs at weeks 1-4, weeks 5-8, and weeks 9-12. The proportion of participants experiencing a ≥75% or 100% response was higher for both doses of atogepant in each 4-week interval assessed. A significantly higher proportion of atogepant-treated participants vs placebo met response criteria at week 12 based on the PGI-C (64.0% and 52.8% vs 35.3%; P<0.001) and satisfaction with study medication (64.6% and 59.2% vs 40.1%; P<0.001). The proportion of atogepant-treated participants experiencing a ≥75% or 100% reduction was greater than placebo at all 4-week intervals with both dosing regimens. The proportion of atogepant-treated participants experiencing a ≥75% or 100% reduction in MMDs increased over each 4-week interval. Table 4a provides PGI-C and Treatment Satisfaction for the mITT participant population. A significantly higher proportion of atogepant-treated participants met response criteria based on the PGI-C and satisfaction with study medication versus placebo.

TABLE 4a
PGI-C and Treatment Satisfaction (mITT population)

| | | Atogepant | Atogepant |
|---|---|---|---|
| Responder Criteria, | Placebo | 30 mg BID | 60 mg QD |
| Week 12 | (n = 246) | (n = 253) | (n = 256) |
| PGI-C,^{a} n/N | 84/238 | 153/239 | 130/246 |
| Percentage | 35.3% | 64.0% | 52.8% |
| OR (95% CI) | — | 3.36 (2.29, 4.94) | 2.16 (1.48, 3.15) |
| P value | | <0.0001 | <0.0001 |
| Satisfaction with study | 91/227 | 148/229 | 138/233 |
| medication,^{b} n/N | | | |
| Percentage | 40.1% | 64.6% | 59.2% |
| OR (95% CI) | — | 2.67 (1.82, 3.91) | 2.16 (1.49, 3.15) |
| P value | | <0.0001 | <0.0001 |

BID, twice daily; mITT, modified intent-to-treat; OR, odds ratio; PGI-C, Patient Global Impression of Change; QD, once daily.
^{a}Response defined as “much better” or “very much better.”
^{b}Response defined as “satisfied” or “extremely satisfied.”

All analyses were performed at the nominal significance level, without adjusting for multiplicity.

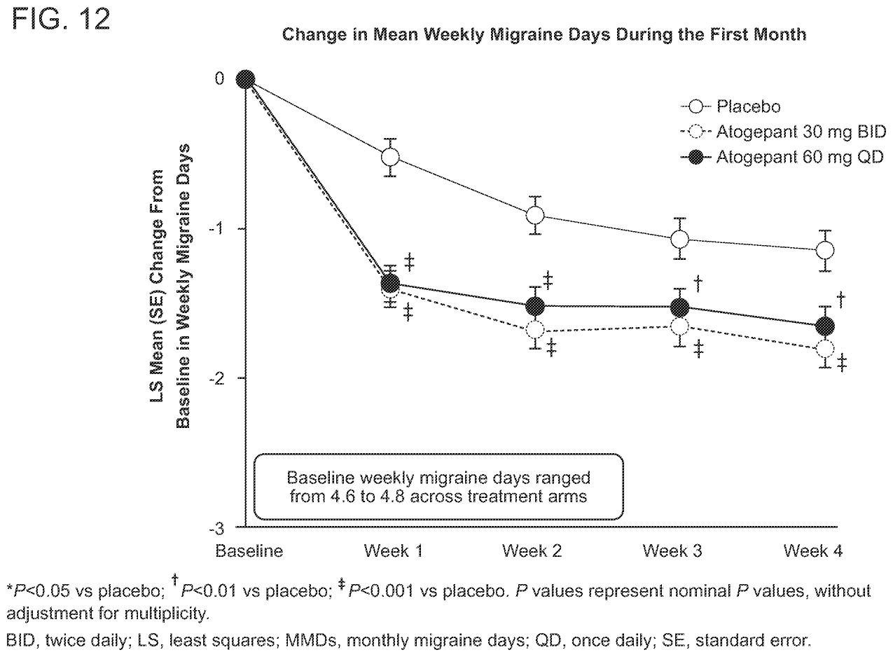

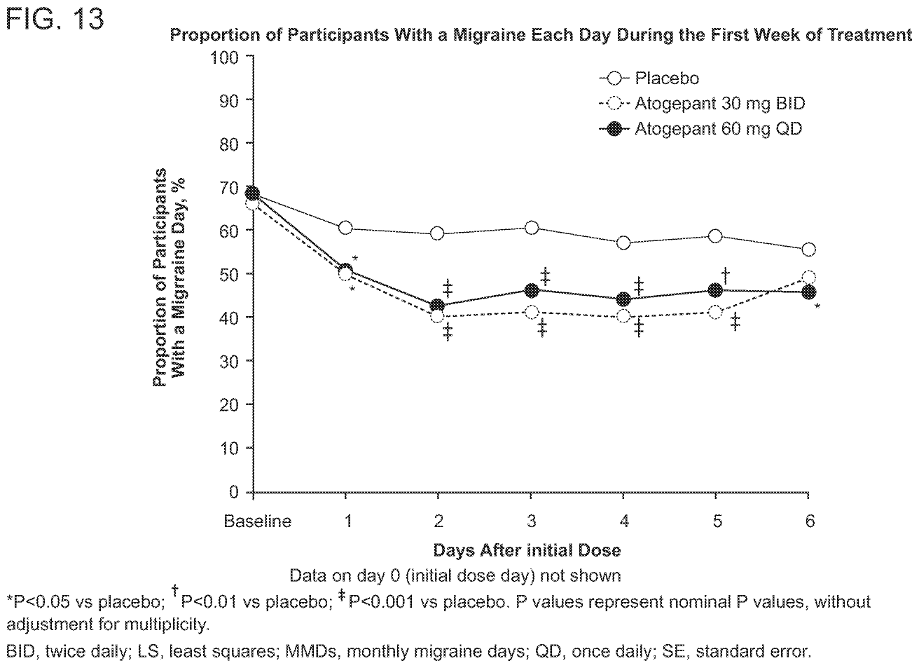

During weeks 1-4 of treatment, a greater reduction in mean MMDs was observed in both atogepant treatment groups compared with placebo. FIG. 12 shows data for patients treated with atogepant 30 mg BID, atogepant 60 mg QD, and placebo who experienced a decrease from baseline in their mean weekly migraine days during the first month of administration. Greater reduction in weekly migraine days was observed in atogepant-treated participants vs placebo during each week of the first month of treatment compared with placebo. FIG. 13 shows the proportion (%) of patients treated with atogepant 30 mg BID, atogepant 60 mg QD, and placebo who experienced a migraine day during the first week of treatment. Efficacy was seen in both atogepant treatment groups as early as the first day after treatment initiation. Treatment-emergent adverse events were reported by 63.2% of participants in the atogepant 60 mg QD arm, 56.4% in the atogepant 30 mg BID arm, and 49.4% in the placebo arm. The most common (≥5%) adverse events in the atogepant groups were constipation (30 mg BID: 28 [11%]; 60 mg QD: 26 [10%]; placebo: 8 [3%]) and nausea (30 mg BID: 20 [8%]; 60 mg QD: 25 [10%]; placebo: 9 [4%]).

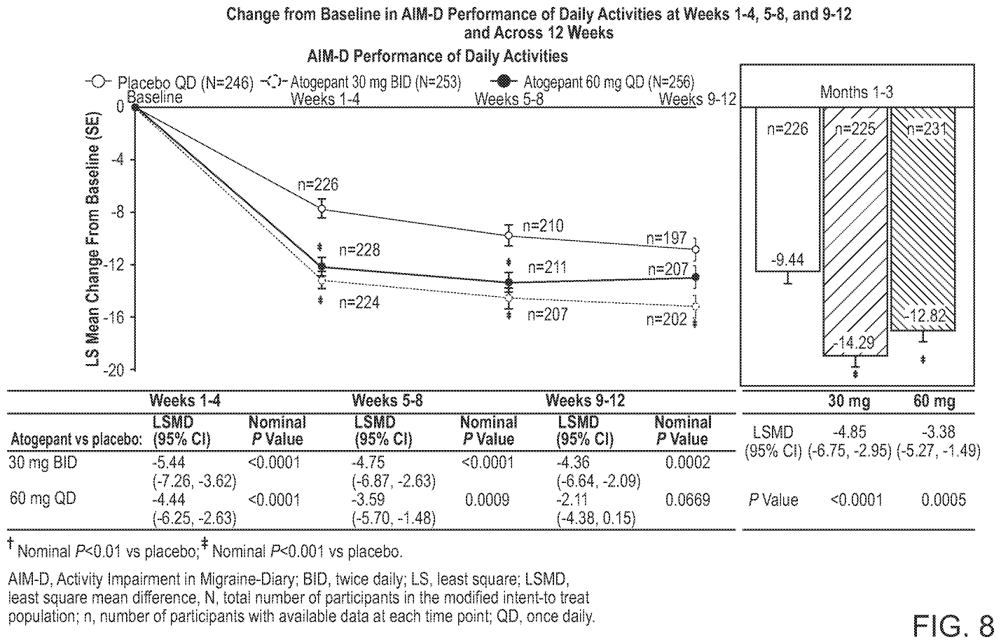

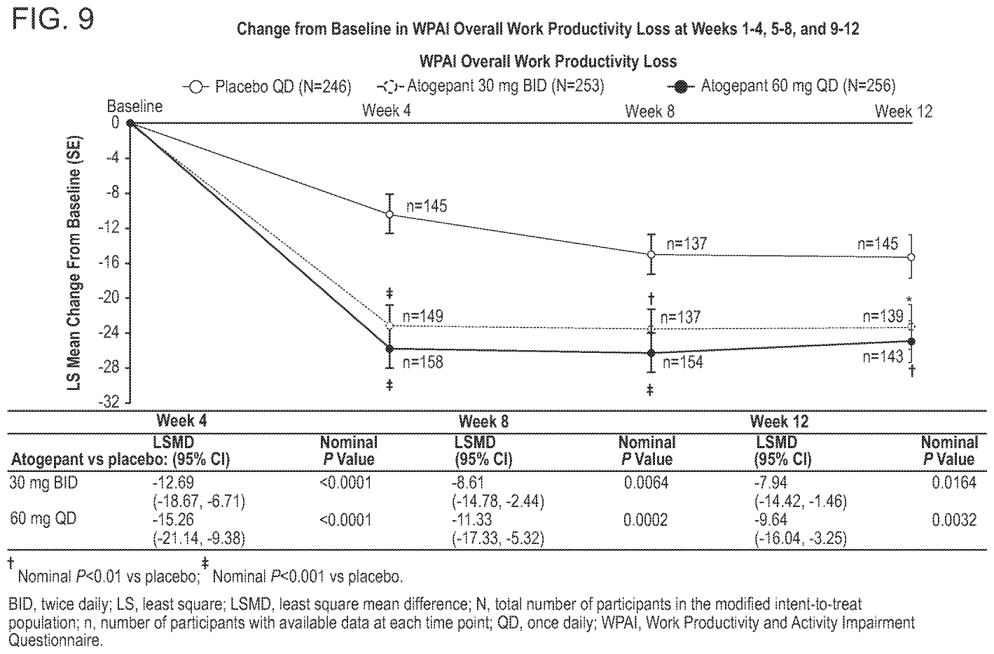

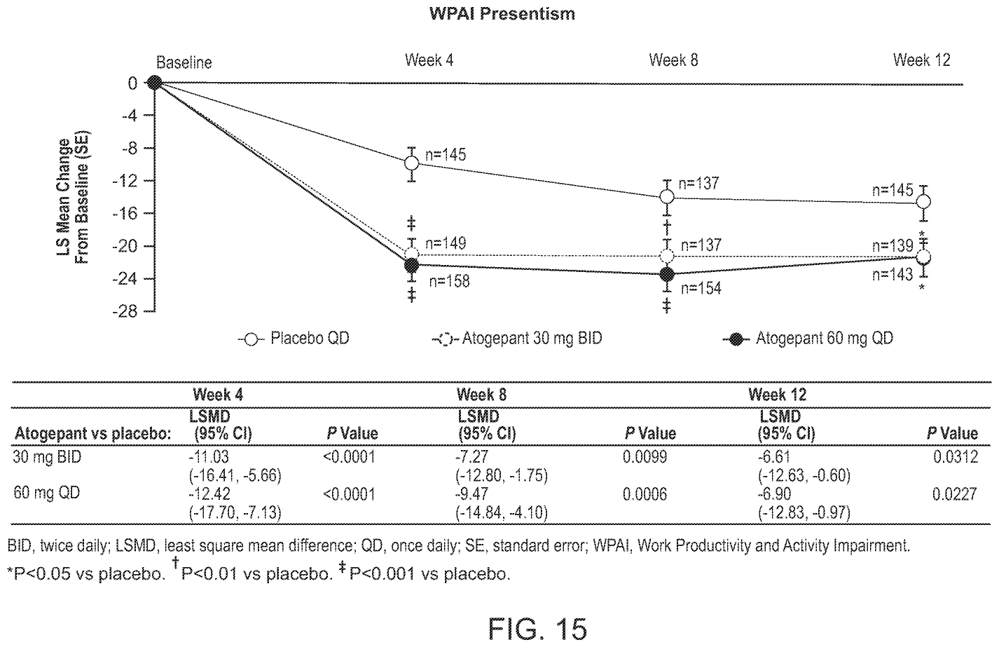

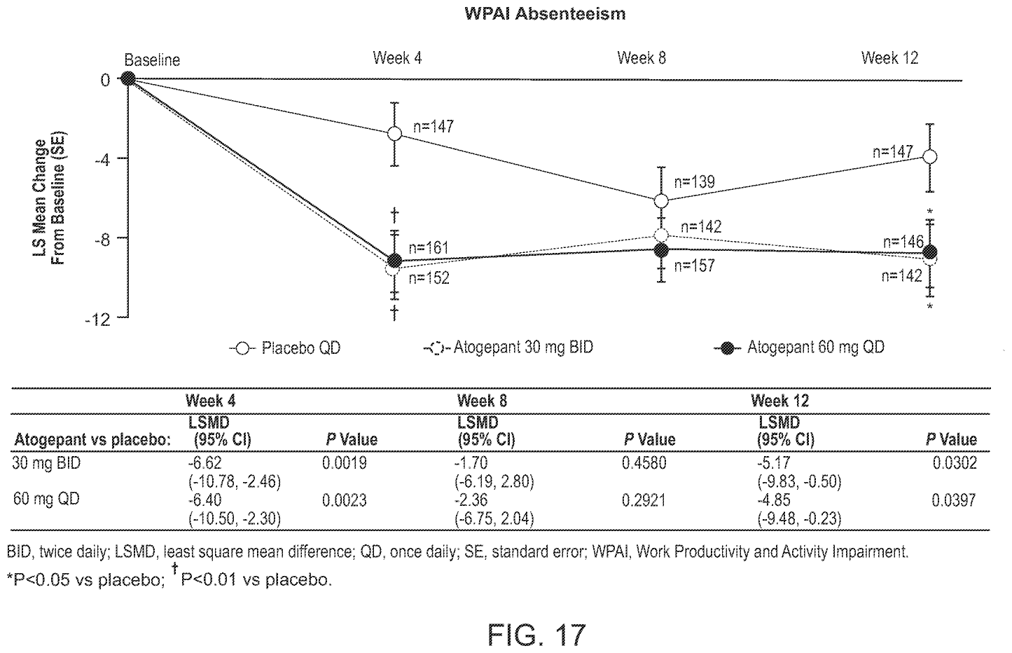

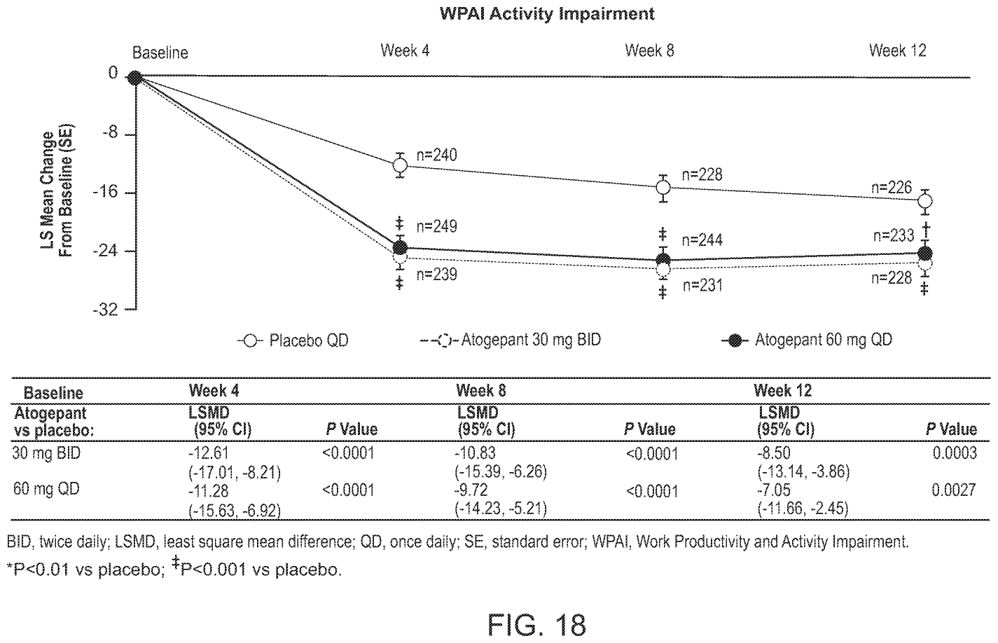

Atogepant demonstrated statistically significant improvements in PRO measures of migraine impact on daily functioning and work productivity. FIG. 8 and Table 5 show the change from baseline in AIM-D Performance of Daily Activities at Weeks 1-4, 5-8, and 9-12 and across 12 weeks. FIG. 9 and Table 6 show change from baseline in WPAI Overall Work Productivity Loss at Weeks 1-4, 5-8, and 9-12. Both doses of atogepant demonstrated statistically significant improvements from baseline across the 12-week treatment period in AIM-D PDA (least squares mean difference [LSMD]: 30 mg BID, −4.85 [95% CI: −6.75, −2.95]; 60 mg QD, −3.38 [−5.27, −1.49]; P<0.001) and PI (LSMD: 30 mg BID, −4.19 [95% CI: −5.95, −2.43]; 60 mg QD, −2.71 [−4.47, −0.96]; P<0.01) domain scores vs placebo. Both atogepant doses demonstrated nominally significant improvements in AIM-D PDA and PI domain scores at weeks 1-4, 5-8, and 9-12 (only for 30 mg BID) vs. placebo. Nominally significant improvements were seen in presenteeism (FIG. 15), overall work productivity loss (FIG. 9), and activity impairment (FIG. 18) for both atogepant doses at all time points, and in absenteeism (FIG. 17) at weeks 4 and 12 for both doses, vs. placebo. Nominally significant improvements were seen in presenteeism (FIG. 15), overall work productivity loss (FIG. 9), and activity impairment (FIG. 18) for both doses (atogepant 30 mg BID and atogepant 60 mg QD) at all time points, and in absenteeism (FIG. 17) at weeks 4 and 12 for both doses, vs placebo (P<0.05). A graphical approach with weighted Bonferroni test procedure was used to control the overall type I error rate at the 2-sided α=0.05 level for key secondary endpoint comparisons between each atogepant dose vs placebo. For exploratory endpoints, nominal P values were provided without adjusting for multiplicity.

TABLE 5
Change from Baseline in AIM-D Performance of Daily Activities at Weeks 1-4, 5-8, and 9-12 and across 12 weeks

| | Weeks 1-4 | | Weeks 5-8 | | Weeks 9-12 | | | | |
|---|---|---|---|---|---|---|---|---|---|
| Atogepant | LSMD | Nominal | LSMD | Nominal | LSMD | Nominal | | | |
| vs placebo | (95% CI) | P Value | (95% CI) | P Value | (95% CI) | P Value | LSMD | 30 mg | 60 mg |
| 30 mg BID | −5.44 | <0.0001 | −4.75 | <0.0001 | −4.36 | 0.0002 | (95% CI) | −4.85 | −3.38 |
| | (−7.26, −3.62) | | (−6.87, −2.63) | | (−6.64, −2.09) | | | (−6.75, −9.25) | (−5.27, , −1.49) |
| 60 mg QD | −4.44 | <0.0001 | −3.59 | 0.0009 | −2.11 | 0.0669 | P Value | <0.0001 | 0.0005 |
| | (−6.25, −2.63) | | (−5.70, −1.48) | | (−4.38, 0.15) | | | | |

TABLE 6
Change from Baseline in WPAI Overall Work Productivity Loss at Weeks 1-4, 5-8, and 9-12

| | Week 4 | | Week 8 | | Week 12 | |
|---|---|---|---|---|---|---|
| Atogepant | LSMD | Nominal | LSMD | Nominal | LSMD | Nominal |
| vs placebo | (95% CI) | P Value | (95% CI) | P Value | (95% CI) | P Value |
| 30 mg BID | −12.69 | <0.0001 | −8.61 | 0.0064 | −7.94 | 0.0164 |
| | (−18.67, −6.71) | | (−14.78, −2.44) | | (−14.42, −1.46) | |
| 60 mg QD | −15.26 | <0.0001 | −11.33 | 0.0002 | −9.64 | 0.0032 |
| | (−21.14, −9.38) | | (−17.33, −5.32) | | (−16.04, −3.25) | |

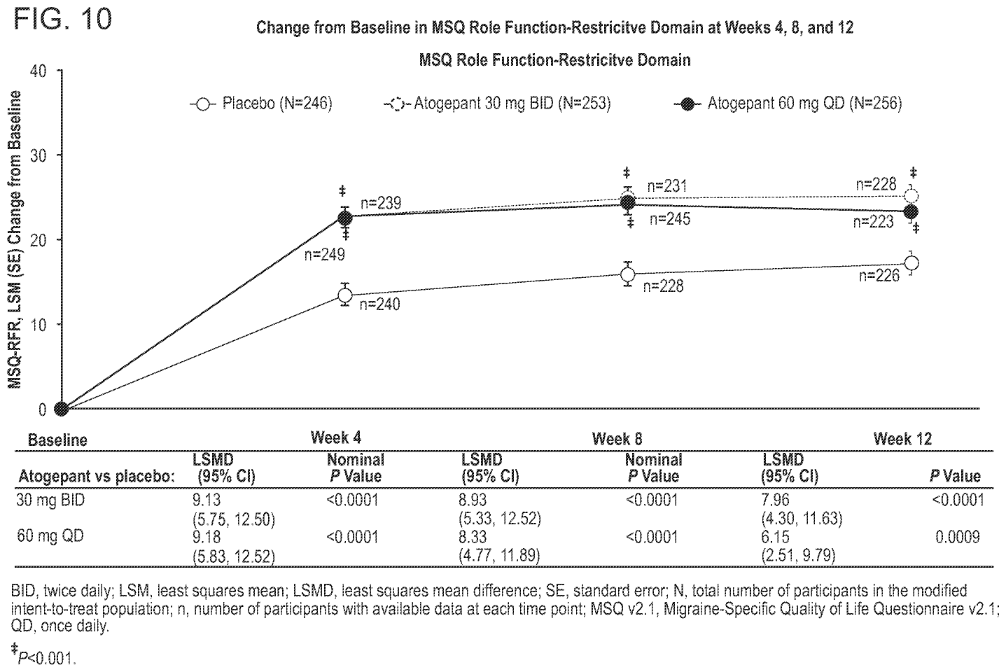

Atogepant demonstrated statistically significant improvements in PRO measures of migraine impact on functional ability and reduction in overall impact of headache. FIG. 10 shows the change from baseline in MSQ Role Function-Restrictive Domain at Weeks 4, 8, and 12. At week 12, increases from baseline (improvements) in all 3 MSQ domain scores were significantly greater in both atogepant treatment arms (30 mg BID and 60 mg QD) compared with placebo (RFR: least squares mean difference [LSMD]: 30 mg BID, 7.96 [95% CI: 4.30, 11.63]; 60 mg QD, 6.15 [2.51, 9.79]; P<0.001; Role Function-Preventive: LSMD: 30 mg BID, 8.10 [95% CI: 4.80, 11.39]; 60 mg QD, 6.86 [3.58, 10.13]; nominal P<0.001; Emotional Function: LSMD: 30 mg BID, 6.36 [95% CI: 2.48, 10.24]; 60 mg QD, 6.81 [2.95, 10.66]; nominal P<0.01).

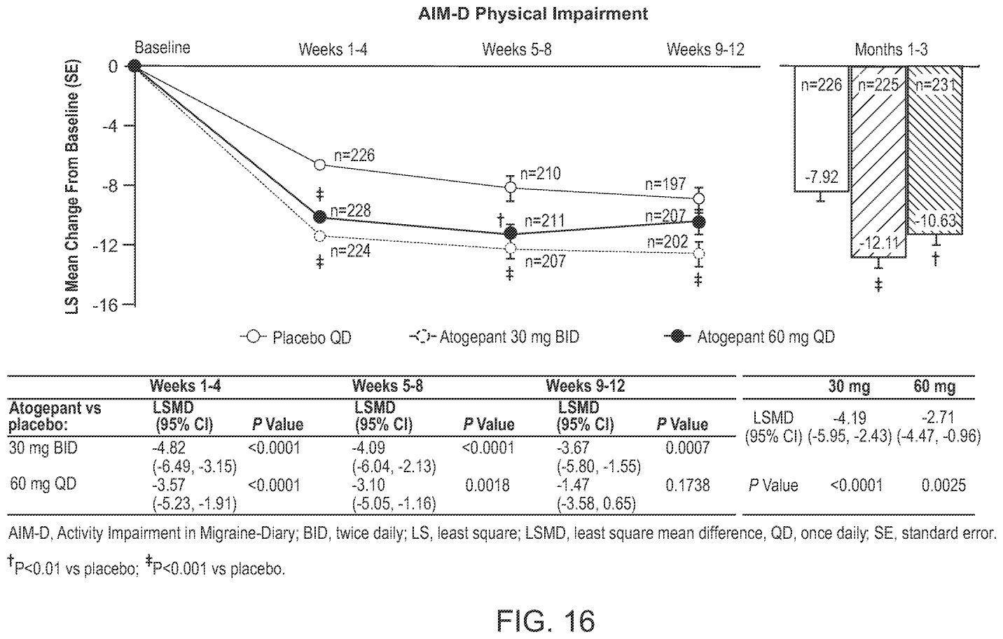

Both doses of atogepant demonstrated statistically significant improvements from baseline in mean monthly AIM-D PDA and PI domain scores across the 12-week treatment period vs placebo. AIM-D PDA least squares mean difference (LSMD): 30 mg BID, 4.85 (95% CI: −6.75, −2.95); 60 mg QD, −3.38 (−5.27, −1.49); P<0.001. AIM-D PI LSMD: 30 mg BID, −4.19 (95% CI: −5.95, −2.43); 60 mg QD, −2.71 (−4.47, −0.96); P<0.01. Both atogepant doses demonstrated nominally significant improvements in AIM-D PDA (FIG. 8) and PI (FIG. 16) domain scores at weeks 1-4, 5-8, and 9-12 (only for 30 mg BID) vs placebo.

Nominally significant improvements were seen in presenteeism (FIG. 15), overall work productivity loss (FIG. 9), and activity impairment (FIG. 18) for both atogepant doses at all time points, and in absenteeism (FIG. 17) at weeks 4 and 12 for both doses, vs placebo (nominal P<0.05).

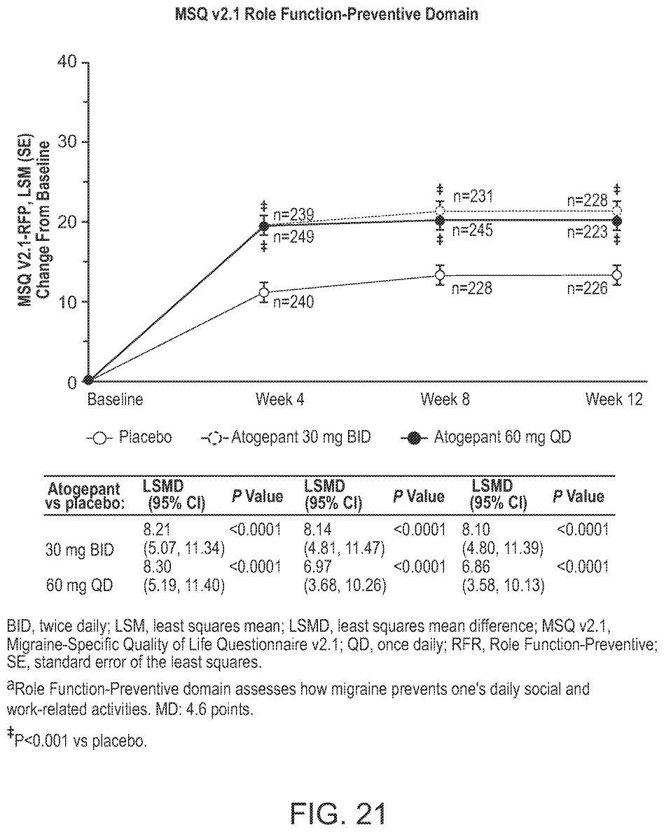

Similar results were seen with all MSQ v2.1 domain scores at weeks 4 and 8 (e.g., FIG. 10). Both atogepant doses demonstrated significant improvement in HIT-6 scores vs. placebo at all assessed time points. Significantly greater proportions of atogepant vs. placebo treated participants were HIT-6 responders (score reduction ≥5 points) at all time points and doses (nominal P<0.001). At week 12, both atogepant treatment arms demonstrated statistically significant improvements from baseline in Role Function-Restrictive MSQ v2.1 domain scores compared with placebo (FIG. 21, least squares mean difference [LSMD]: 30 mg BID, 7.96 [95% CI: 4.30, 11.63]; 60 mg QD, 6.15 [2.51, 9.79]; P<0.001). Nominally significant improvements were seen at weeks 4 and 8 (FIG. 10).

At week 12, both atogepant treatment arms demonstrated nominally significant improvements from baseline in Role Function-Preventive (LSMD: 30 mg BID, 8.10 [95% CI: 4.80, 11.39]; 60 mg QD, 6.86 [3.58, 10.13]; nominal P<0.001) and Emotional Function (LSMD: 30 mg BID, 6.36 [95% CI: 2.48, 10.24]; 60 mg QD, 6.81 [2.95, 10.66]; nominal P<0.01) MSQ v2.1 domain scores compared with placebo. Similar results were seen at weeks 4 and 8 (FIG. 21).

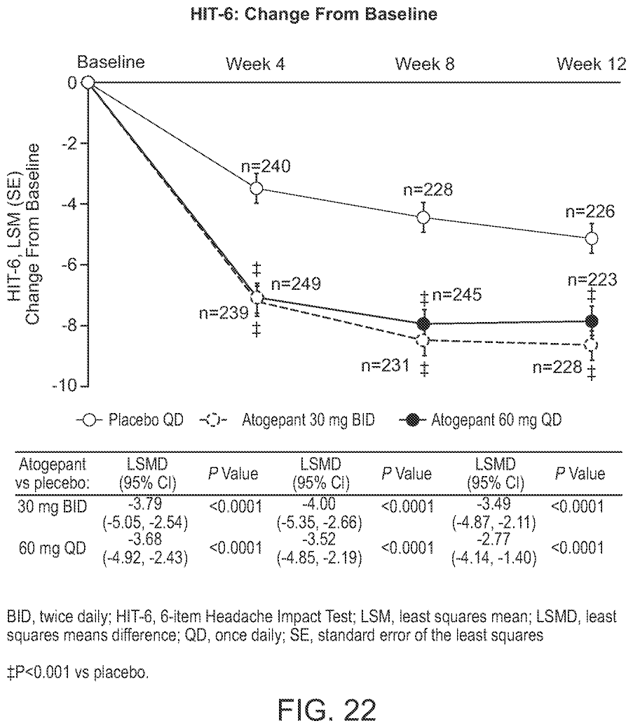

As shown in FIG. 22, both atogepant doses demonstrated a statistically significant improvement in HIT-6 scores vs placebo at week 12 (P<0.001). Both atogepant doses demonstrated nominally significant improvements in HIT-6 scores vs placebo at weeks 4 and 8 (nominal P<0.001). The between-group minimal important difference (≥2.3 points) was achieved at the earliest time point assessed (week 4) and throughout the double-blind period for both atogepant doses. The proportion of atogepant- vs placebo-treated participants who met HIT-6 responder criteria (score reduction ≥5 points) was nominally significantly greater at all time points and doses (nominal P<0.001).

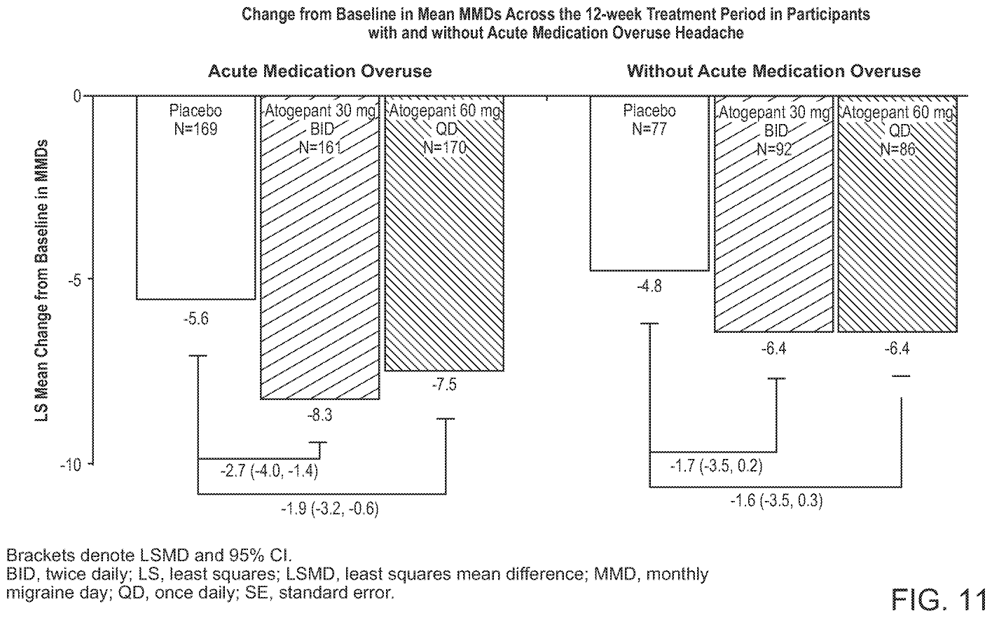

With respect to acute medication overuse, from 778 participants randomized to treatment, and 755 participants in the modified intent-to-treat population, 500 (66.2%) had acute medication overuse at baseline, placebo: n=169 (68.7%); atogepant 30 mg BID: n=161 (63.6%); atogepant 60 mg QD: n=170 (66.4%), where acute medication overuse refers to either use of triptans for ≥10 days/month, use of ergots for ≥10 days/month, use of simple analgesics for ≥15 days/month, or use of any combination of triptans, ergots, or simple analgesics for ≥10 days /month. Participants with use of opioid and/or barbiturates >4 days/month were excluded. Outcomes assessed included the change from baseline in mean MMDs and mean monthly acute medication use days, and ≥50% reduction in mean MMDs across 12 weeks. Among those with acute medication overuse at baseline, the least squares mean (LSM) change from baseline in mean MMDs in atogepant-treated participants (30 mg BID and 60 mg QD) was −8.3 and −7.5 compared with −5.6 in the placebo arm. FIG. 11 shows the change from baseline in MMDs across the 12-week treatment period in participants with and without acute medication overuse headache. The LSM change from baseline in mean monthly acute medication use days in atogepant-treated acute medication over-users was −8.4 and −8.1 compared with −5.5 in the placebo arm (LSM difference [95% CI]: −2.8 [−4.1, −1.6] and −2.6 [−3.9, −1.3], respectively). Additionally, a ≥50% reduction in mean MMDs across the 12-week treatment period was achieved by a nominally significant greater proportion of acute medication overuse participants treated with atogepant 30 mg BID (44.7%) and atogepant 60 mg QD (41.8%) compared with placebo (24.9%; P<0.001). Regardless of acute medication overuse at baseline, atogepant was shown to be effective and was associated with a greater reduction in acute medication use days people with CM.

Table 7 presents the analysis results for the primary and secondary endpoints in the Analysis Population for Off-Treatment Hypothetical Estimand after multiplicity adjustment. Participants in the Off-Treatment Hypothetical Estimand population who started a new migraine prophylaxis treatment during the double-blind or safety follow-up period had their data during the follow-up period starting after the new migraine prophylaxis treatment excluded from the analysis, while those who discontinued study treatment due to all reasons other than starting a new migraine prophylaxis treatment had their data collected after discontinuation of study treatment, and those off-treatment data were included in the analysis. Results for the primary efficacy endpoint and all key secondary endpoints demonstrated statistically significant treatment differences between each atogepant dose group.

TABLE 7
Summary of Primary and Secondary Efficacy Analyses with Multiplicity
Adjustment - Analysis Population for Off-Treatment Hypothetical Estimand

| | | Atogepant | Atogepant |
|---|---|---|---|
| | Placebo | 30 mg BID | 60 mg QD |
| Key Efficacy Endpoints | (N = 249) | (N = 254) | (N = 257) |
| P1: Change from baseline in mean monthly | | | |
| migraine days across the 12-week treatment | | | |
| period | | | |
| Baseline number of monthly migraine days, | 18.95 | 18.60 | 19.19 |
| Mean (SD) | (4.795) | (5.090) | (5.291) |
| LS Mean (SE) | −5.09 | −7.33 | −6.75 |
| | (0.409) | (0.406) | (0.406) |
| LSMD (95% CI), Atogepant vs. Placebo | | −2.24 | −1.66 |
| | | (−3.31, −1.16) | (−2.72, −0.59) |
| Nominal p-value from model | | <0.0001 | 0.0024 |
| Adjusted p-value | | 0.0001 | 0.0024 |
| S1: Change from baseline in mean monthly | | | |
| headache days across the 12-week treatment | | | |
| period | | | |
| Baseline number of monthly headache days, | 21.42 | 21.17 | 21.54 |
| Mean (SD) | (4.111) | (4.145) | (4.323) |
| LS Mean (SE) | −5.17 | −7.32 | −6.90 |
| | (0.403) | (0.399) | (0.399) |
| LSMD (95% CI), Atogepant vs. Placebo | | −2.14 | −1.72 |
| | | (−3.20, −1.09) | (−2.78, −0.67) |
| Nominal p-value from model | | <0.0001 | 0.0014 |
| Adjusted p-value | | 0.0002 | 0.0024 |
| S2: Change from baseline in mean monthly acute | | | |
| medication use days across the 12-week treatment | | | |
| period | | | |
| Baseline number of monthly acute medication use | 15.31 | 14.53 | 15.45 |
| days, Mean (SD) | (7.048) | (7.223) | (7.363) |
| LS Mean (SE) | −4.09 | −6.61 | −6.19 |
| | (0.389) | (0.388) | (0.383) |
| LSMD (95% CI), Atogepant vs. Placebo | | −2.52 | −2.09 |
| | | (−3.52, −1.53) | (−3.09, −1.10) |
| Nominal p-value from model | | <0.0001 | <0.0001 |
| Adjusted p-value | | 0.0002 | 0.0024 |
| S3: >=50% reduction in 3-month average of | | | |
| monthly migraine days | | | |
| Responders, n (%) | 66 (26.5) | 107 (42.1) | 103 (40.1) |
| Odds ratio (95% CI), Atogepant vs. Placebo | | 2.03 | 1.90 |
| | | (1.38, 2.98) | (1.29, 2.79) |
| Nominal p-value from model | | 0.0003 | 0.0011 |
| Adjusted p-value | | 0.0006 | 0.0024 |
| S4: Change from Baseline in HIT-6 total score at | | | |
| Week 12 | | | |
| Baseline HIT-6 total score, Mean (SD) | 63.96 | 64.25 | 64.22 |
| | (4.797) | (5.191) | (5.053) |
| LS Mean (SE) | −5.17 | −8.47 | −7.83 |
| | (0.515) | (0.512) | (0.507) |
| LSMD (95% CI), Atogepant vs. Placebo | | −3.30 | −2.66 |
| | | (−4.67, −1.93) | (−4.02, −1.30) |
| Nominal p-value from model | | <0.0001 | 0.0001 |
| Adjusted p-value | | 0.0006 | 0.0024 |
| S5: Change from Baseline in MSQ v2.1 Role | | | |
| Function - Restrictive Domain Score at Week 12 | | | |
| Baseline MSQ v2.1 role function restrictive domain | 43.97 | 43.98 | 43.64 |
| score, Mean (SD) | (19.078) | (18.979) | (18.973) |
| LS Mean (SE) | 17.30 | 24.74 | 23.09 |
| | (1.378) | (1.371) | (1.359) |
| LSMD (95% CI), Atogepant vs. Placebo | | 7.43 | 5.78 |
| | | (3.77, 11.09) | (2.15, 9.41) |
| Nominal p-value from model | | <0.0001 | 0.0018 |
| Adjusted p-value | | 0.0006 | 0.0024 |

Across the 12 weeks, based on the OTHE population, patients in the 60 mg QD and 30 mg BID atogepant treatment arms of the study experienced a decrease of 6.75 and 7.33 monthly migraine days, respectively, compared to patients in the placebo arm, who experienced a decrease of 5.09 monthly migraine days (60 mg QD vs. placebo, p=0.0024; 30 mg BID vs. placebo, p=0.0001, adjusted for multiple comparisons).

From the safety population, 760 individuals were included in the off-treatment hypothetical estimand population and 269 were included in the Europe subpopulation (Placebo n=88, Atogepant 30 mg BID n=91, atogepant 60 mg QD n=90). Least square (LS) mean change in MMDs was −8.44 in the atogepant 30 mg BID and −8.00 in the atogepant 60 mg QD subgroups compared to −5.42 in the placebo group. LS mean difference [95% CI] vs. placebo was greater in both groups (atogepant 30 mg BID: −3.02 [−4.82, −1.22]; atogepant 60 mg QD: −2.59 [−4.39, −0.79]). A higher proportion of atogepant 30 mg BID (48.4%; OR [95% CI]: 1.87 [1.01, 3.44]; nominal P=0.0457) and atogepant 60 mg QD (46.7%; OR [95% CI]: 1.84 [1.00, 3.41]; nominal P=0.0511) participants had a ≥50% reduction in 3-month average of MMDs compared to placebo (33.0%).

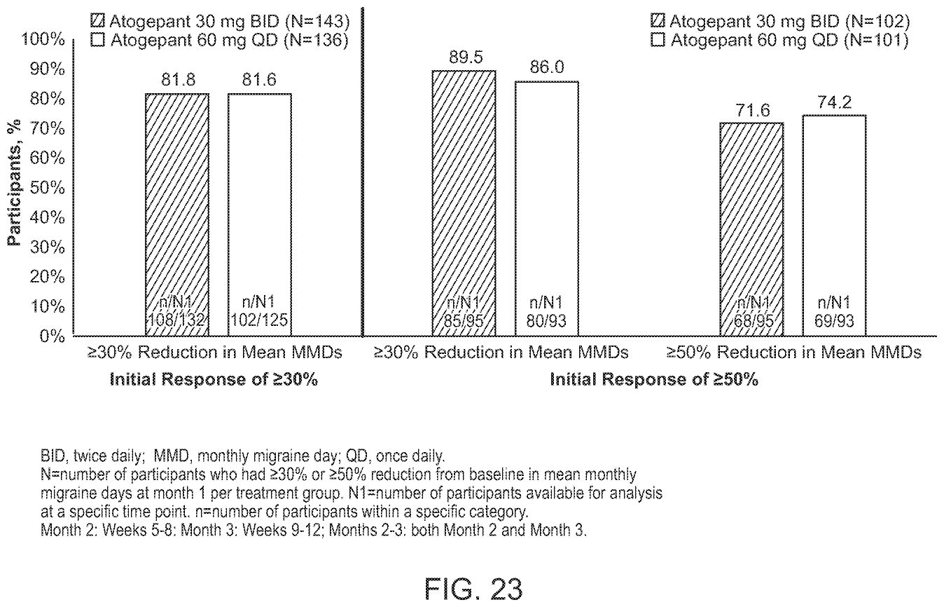

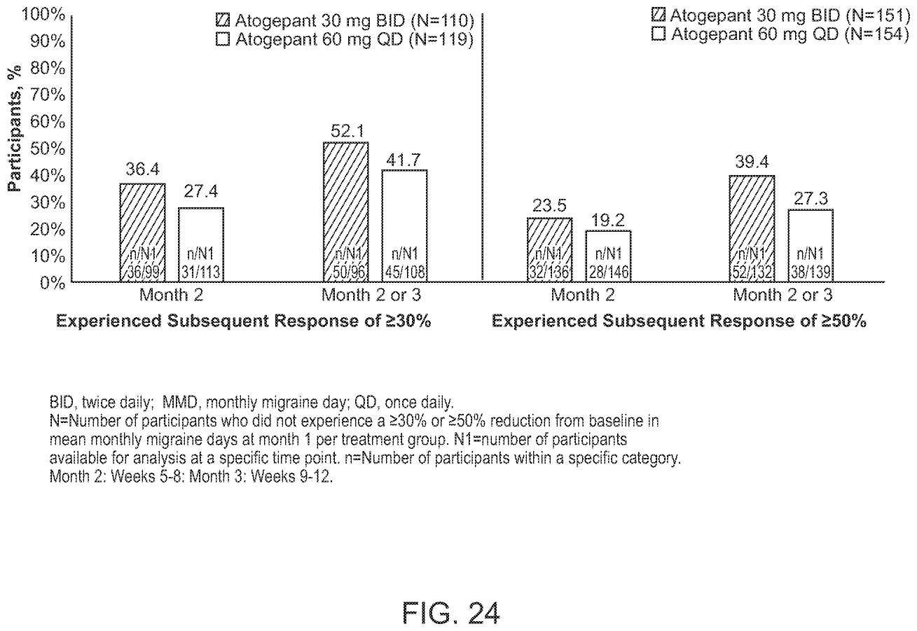

Notably, most participants who experienced an initial response during month 1 continued to experience a ≥30% or ≥50% response throughout the 12-week treatment period. While a substantial proportion of atogepant-treated participants in the trial experienced treatment response within the first month, of those who did not, a notable proportion experienced a ≥30% or ≥50% reduction in MMDs in the second or third month. Sustained response was calculated by evaluating the proportion of participants who had an initial response in month 1 and continued to experience at least that degree of response in months 2 and 3. Initial response thresholds of ≥30%, ≥50%, and ≥75% reduction from baseline in mean MMDs were evaluated. Subsequent response was calculated by evaluating the proportion of atogepant-treated participants who experienced <30% or <50% reduction from baseline in mean MMDs in month 1 who then experienced ≥30% or ≥50% reduction from baseline in mean MMDs in month 2 and in either month 2 or month 3, as well as the proportion of atogepant-treated participants who experienced <30% or <50% reduction in mean MMDs in both month 1 and month 2 who then experienced ≥30% or ≥50% reduction in mean MMDs in month 3. For these analyses, a month was defined as each 4-week treatment interval (i.e., weeks 1-4, 5-8, and 9-12). For this analysis, a total of 755 participants (mean age: 42.1 years; 87.5% female) were evaluable for efficacy and included in the mITT population (atogepant 30 mg twice daily, n=253; atogepant 60 mg once daily, n=256). Of participants who experienced ≥30% or ≥50% response in month 1, most experienced a sustained response of ≥30% (atogepant 30 mg twice daily: 81.8% [108/132]; 60 mg once daily: 81.6% [102/125]) or ≥50% (atogepant 30 mg twice daily: 71.6% [68/95]; 60 mg once daily: 74.2% [69/93]) throughout the trial (FIG. 23). Of participants who experienced ≥75% response in month 1, 46.9% (23/49) in the 30 mg twice daily and 52.5% (21/40) in the 60 mg once daily group experienced a sustained response throughout the trial. Among participants with <30% reduction from baseline in mean MMDs in month 1, 27.4%-36.4% experienced ≥30% reduction in mean MMDs in month 2 (atogepant 30 mg twice daily, 36/99; 60 mg once daily, 31/113) and 41.7%-52.1% experienced ≥30% reduction in mean MMDs in either month 2 or 3 (atogepant 30 mg twice daily, 50/96; 60 mg once daily, 45/108; FIG. 24). Among participants with <50% reduction from baseline in mean MMDs in month 1, 19.2%-23.5% experienced ≥50% reduction in mean MMDs in month 2 (atogepant 30 mg twice daily, 32/136; 60 mg once daily, 28/146) and 27.3%-39.4% experienced ≥50% reduction in mean MMDs in either month 2 or 3 (atogepant 30 mg twice daily, 52/132; 60 mg once daily, 38/139; FIG. 24). A number of participants who did not experience ≥30% reduction in mean MMDs in months 1 or 2 experienced a ≥30% response by the end of the trial (atogepant 30 mg twice daily, 23.3% [14/60]; 60 mg once daily, 17.1% [13/76]). A number of participants who did not experience ≥50% reduction in mean MMDs in months 1 or 2 experienced a ≥50% response by the end of the trial (atogepant 30 mg twice daily, 20.0% [20/100]; 60 mg once daily, 8.2% [9/110]).

The study demonstrated that treatment with atogepant 60 mg QD and 30 mg BID resulted in statistically and clinically significant improvements in all primary and secondary endpoints for both efficacy analysis populations.

The overall safety profile of the study was consistent with safety findings observed in previous studies of atogepant in an episodic migraine population. Each dose of atogepant was generally well tolerated. For the safety population, the mean treatment duration for atogepant 30 mg BID, atogepant 60 mg QD, and placebo group were 80.1 days, 79.7 days, and 80.3 days, respectively. Serious adverse events occurred in 2.7% of patients treated with atogepant 60 mg QD and 1.6% of patients treated with atogepant 30 mg BID, compared to 1.2% of patients with placebo. The most common adverse events reported with a frequency ≥5% in at least one atogepant treatment arm, and greater than placebo, were constipation (10.0% for atogepant 60 mg QD, 10.9% for atogepant 30 mg BID, and 3.1% for placebo) and nausea (9.6% for atogepant 60 mg QD, 7.8% for atogepant 30 mg BID, and 3.5% for placebo).

Adverse events for the safety population are summarized in Table 8.

TABLE 8
Overall Summary of Adverse Events - Safety Population

| | | Atogepant | Atogepant |
|---|---|---|---|
| | Placebo | 30 mg BID | 60 mg QD |
| | (N = 255) | (N = 257) | (N = 261) |
| | n (%) | n (%) | n (%) |
| TEAEs | 126 (49.4) | 145 (56.4) | 165 (63.2) |
| Treatment-related TEAEs | 34 (13.3) | 52 (20.2) | 45 (17.2) |
| Deaths | 0 | 0 | 0 |
| Treatment-emergent SAEs | 3 (1.2) | 4 (1.6) | 7 (2.7) |
| (TESAE) | | | |
| TEAE leading to treatment | 10 (3.9) | 13 (5.1) | 9 (3.4) |
| discontinuation | | | |

The treatment emergent adverse events that occurred in >=2% participants in at least one treatment group are summarized in Table 9. The incidences rates of constipation, nausea, dizziness, decreased appetite, fatigue, urinary tract infection, abdominal pain, and back pain were greater in both doses than placebo. The incidence rate of abdominal pain—upper was greater than placebo in the atogepant 30 mg BID group. The incidence of pyrexia was greater than placebo in the atogepant 60 mg QD group.

TABLE 9
≥2% Treatment Emergent Adverse Events - Safety Population

| | | Atogepant | | Atogepant | |
|---|---|---|---|---|---|
| | Placebo | 30 mg BID | | 60 mg QD | |
| | (N = 255) | (N = 257) | | (N = 261) | |
| | n (%) | n (%) | | n (%) | |
| Constipation | 8 (3.1) | 28 | (10.9) | 26 | (10.0) |
| Nausea | 9 (3.5) | 20 | (7.8) | 25 | (9.6) |
| Dizziness | 8 (3.1) | 11 | (4.3) | 12 | (4.6) |
| Nasopharyngitis | 11 (4.3) | 10 | (3.9) | 11 | (4.2) |
| Decreased appetite | 0 | 7 | (2.7) | 9 | (3.4) |
| Fatigue | 7 (2.7) | 11 | (4.3) | 8 | (3.1) |
| Pyrexia | 3 (1.2) | 2 | (0.8) | 8 | (3.1) |
| Urinary tract infection | 3 (1.2) | 10 | (3.9) | 6 | (2.3) |
| Abdominal pain | 3 (1.2) | 6 | (2.3) | 5 | (1.9) |
| Diarrhoea | 6 (2.4) | 2 | (0.8) | 5 | (1.9) |
| Insomnia | 5 (2.0) | 5 | (1.9) | 5 | (1.9) |
| COVID-19 | 5 (2.0) | 4 | (1.6) | 4 | (1.5) |
| Migraine | 5 (2.0) | 3 | (1.2) | 4 | (1.5) |
| Abdominal pain upper | 5 (2.0) | 7 | (2.7) | 3 | (1.1) |
| Arthralgia | 6 (2.4) | 5 | (1.9) | 3 | (1.1) |
| Back pain | 0 | 6 | (2.3) | 3 | (1.1) |
| Upper respiratory tract infection | 6 (2.4) | 6 | (2.3) | 2 | (0.8) |

Treatment emergent serious adverse events for the safety population are summarized in Table 10. None of the serious adverse events were considered related to study treatment by the investigator.

TABLE 10
Treatment Emergent Serious Adverse Events - Safety Population

| | | Atogepant | Atogepant |
|---|---|---|---|
| | Placebo | 30 mg BID | 60 mg QD |
| System Organ Class | (N = 255) | (N = 257) | (N = 261) |
| Preferred Term | n (%) | n (%) | n (%) |
| Participants with at least one | 3 (1.2) | 4 (1.6) | 7 (2.7) |
| TESAE | | | |
| Cholelithiasis | 0 | 0 | 1 (0.4) |
| COVID-19 | 0 | 0 | 1 (0.4) |
| Anal abscess | 0 | 1 (0.4) | 0 |
| COVID-19 pneumonia | 0 | 1 (0.4) | 0 |
| Fall | 0 | 0 | 1 (0.4) |
| Hip fracture | 0 | 0 | 1 (0.4) |
| Road traffic accident | 0 | 0 | 1 (0.4) |
| Vaccination complication | 0 | 0 | 1 (0.4) |
| Epicondylitis | 1 (0.4) | 0 | 0 |
| Spinal pain | 0 | 0 | 1 (0.4) |
| Spinal cord neoplasm | 0 | 0 | 1 (0.4) |
| Benign ovarian tumour | 0 | 1 (0.4) | 0 |
| Plasma cell myeloma | 1 (0.4) | 0 | 0 |
| Suicide attempt | 1 (0.4) | 1 (0.4) | 0 |
| Nasal septum deviation | 0 | 0 | 1 (0.4) |

Table 11 presents the number and percentage of participants with post-baseline hepatic-related laboratory parameter values of clinical interest. There were five participants who had post-baseline ALT or AST elevations (≥3*ULN), with 1/254 (0.4%), 2/255 (0.8%), and 2/257 (0.8%) in the placebo, atogepant 30 mg BID, and atogepant 60 mg QD groups, respectively. All cases in the atogepant dose groups were adjudicated as unlikely to be related to study drug. No participants met potential Hy's law criteria. No hepatic safety issues related to atogepant were identified.

TABLE 11
Number of Participants with Post-Baseline Hepatic-Related
Laboratory Values of Clinical Interest - Safety Population

| | | | Atogepant | | Atogepant | |
|---|---|---|---|---|---|---|
| | Placebo | | 30 mg BID | | 60 mg QD | |
| Parameter (Unit) | (N = 255) | | (N = 257) | | (N = 261) | |
| Criterion | n/N1 (%) | | n/N1 (%) | | n/N1 (%) | |
| ALT (U/L) | | | | | | |
| >=1 * ULN | 28/254 | (11.0) | 18/255 | (7.1) | 17/257 | (6.6) |
| >=1.5 * ULN | 12/254 | (4.7) | 7/255 | (2.7) | 6/257 | (2.3) |
| >=2 * ULN | 5/254 | (2.0) | 2/255 | (0.8) | 2/257 | (0.8) |
| >=3 * ULN | 1/254 | (0.4) | 2/255 | (0.8) | 1/257 | (0.4) |
| >=5 * ULN | 0 | | 1/255 | (0.4) | 1/257 | (0.4) |
| >=10 * ULN | 0 | | 0 | | 0 | |
| >=20 * ULN | 0 | | 0 | | 0 | |
| AST (U/L) | | | | | | |
| >=1 * ULN | 19/254 | (7.5) | 10/255 | (3.9) | 8/257 | (3.1) |
| >=1.5 * ULN | 6/254 | (2.4) | 5/255 | (2.0) | 3/257 | (1.2) |
| >=2 * ULN | 2/254 | (0.8) | 3/255 | (1.2) | 2/257 | (0.8) |
| >=3 * ULN | 0 | | 1/255 | (0.4) | 2/257 | (0.8) |
| >=5 * ULN | 0 | | 0 | | 2/257 | (0.8) |
| >=10 * ULN | 0 | | 0 | | 0 | |
| >=20 * ULN | 0 | | 0 | | 0 | |
| ALT or AST (U/L) | | | | | | |
| >=1 * ULN | 30/254 | (11.8) | 21/255 | (8.2) | 18/257 | (7.0) |
| >=1.5 * ULN | 13/254 | (5.1) | 8/255 | (3.1) | 6/257 | (2.3) |
| >=2 * ULN | 6/254 | (2.4) | 3/255 | (1.2) | 3/257 | (1.2) |
| >=3 * ULN | 1/254 | (0.4) | 2/255 | (0.8) | 2/257 | (0.8) |
| >=5 * ULN | 0 | | 1/255 | (0.4) | 2/257 | (0.8) |
| >=10 * ULN | 0 | | 0 | | 0 | |
| >=20 * ULN | 0 | | 0 | | 0 | |
| Bilirubin Total (umol/L) | | | | | | |
| >=1 * ULN | 9/254 | (3.5) | 8/255 | (3.1) | 11/257 | (4.3) |
| >=1.5 * ULN | 2/254 | (0.8) | 1/255 | (0.4) | 1/257 | (0.4) |
| >=2 * ULN | 0 | | 0 | | 0 | |
| >=3 * ULN | 0 | | 0 | | 0 | |
| >=5 * ULN | 0 | | 0 | | 0 | |
| >=10 * ULN | 0 | | 0 | | 0 | |
| >=20 * ULN | 0 | | 0 | | 0 | |
| Alkaline Phosphatase (U/L) | | | | | | |
| >=1 * ULN | 16/254 | (6.3) | 16/255 | (6.3) | 18/257 | (7.0) |

| | | | | | |
|---|---|---|---|---|---|
| >=1.5 * ULN | 0 | 2/255 | (0.8) | 1/257 | (0.4) |

| | | | | |
|---|---|---|---|---|
| >=2 * ULN | 0 | 1/255 | (0.4) | 0 |
| >=3 * ULN | 0 | 1/255 | (0.4) | 0 |

| | | | |
|---|---|---|---|
| >=5 * ULN | 0 | 0 | 0 |
| >=10 * ULN | 0 | 0 | 0 |
| >=20 * ULN | 0 | 0 | 0 |
| Concurrent Elevations | | | |
| ALT or AST >= 3 * ULN AND | 0 | 0 | 0 |
| Bilirubin Total >= 1.5 * ULN | | | |
| ALT or AST >= 3 * ULN AND | 0 | 0 | 0 |
| Bilirubin Total >= 2 * ULN | | | |
| Potential Hy's Law | 0 | 0 | 0 |
| ALT or AST >= 3 * ULN AND | | | |
| Bilirubin Total >= 2 * ULN AND | | | |
| ALP < 2 * ULN | | | |

Atogepant 30 mg BID and 60 mg QD groups showed statistically significant improvement over placebo group in the primary efficacy endpoint and all key secondary efficacy endpoints. Both doses were well tolerated, and the safety results were consistent with the safety profile of atogepant.

### Example 2

Analysis of diary-based patient-reported outcome data from the trial disclosed herein suggests that atogepant provides rapid improvements in daily functioning, physical impairment, and quality of life (QOL) relative to placebo. Effective preventive treatments for chronic migraine (CM) should reduce migraine symptoms and attack frequency and improve functioning and QOL soon after initiating treatment. Analysis of data obtained from the trial was used to evaluate the impact of the early onset of effect of atogepant on functioning and QOL in CM.

Patient-reported outcome (PRO) measures included the Performance of Daily Activities (PDA) and Physical Impairment (PI) domains of the Activity Impairment in Migraine-Diary (AIM-D), and the 5-level European Quality of Life-5 Dimension (EQ-5D-5L) descriptive system and visual analogue scale (VAS), implemented via daily diary. Scores in each domain were transformed to a 0-100 scale with higher scores indicating a greater burden from migraine. Changes in PDA and PI scores and the proportion of participants achieving clinically meaningful within-patient change in the PDA domain (≥12.5 points) and PI domain (≥10 points) were compared between participants who received atogepant vs placebo. EQ-5D-5L measures 5 dimensions of QOL, including mobility, self-care, usual activities, pain/discomfort, and anxiety/depression. Index scores range from 0-1 with scores closer to 1 indicating a better state of health. EQ VAS uses a vertical scale ranging from 0 (“the worst health you can imagine”) to 100 (“the best health you can imagine”). Changes from baseline in weekly AIM-D PDA and PI scores were calculated for weeks 1, 2, 3, and 4. Changes from baseline in EQ-5D-5L descriptive system were evaluated for weeks 1-2 and at week 4 (week 3 data not collected) using diary data at these time points in the trial.

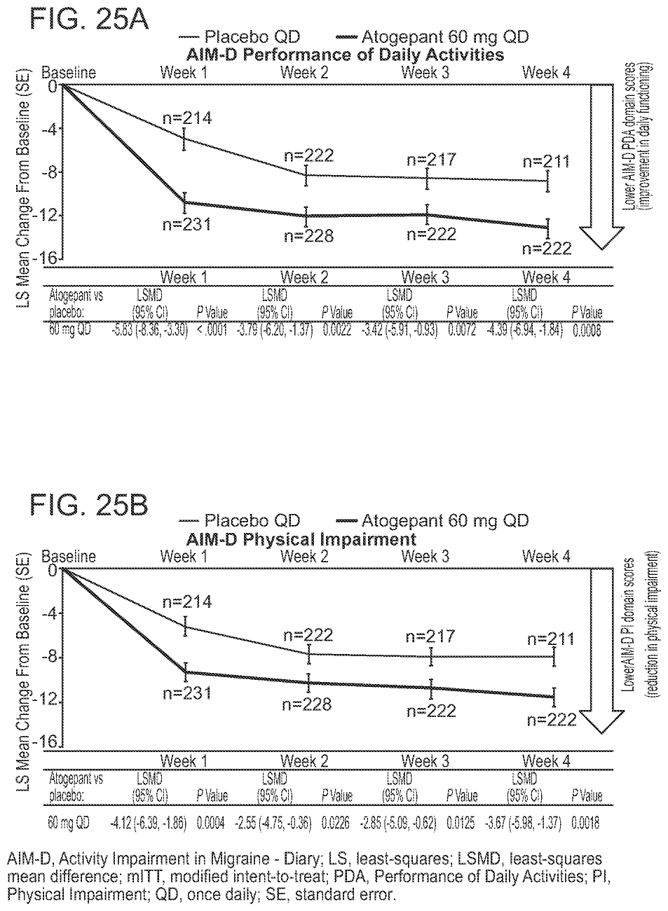

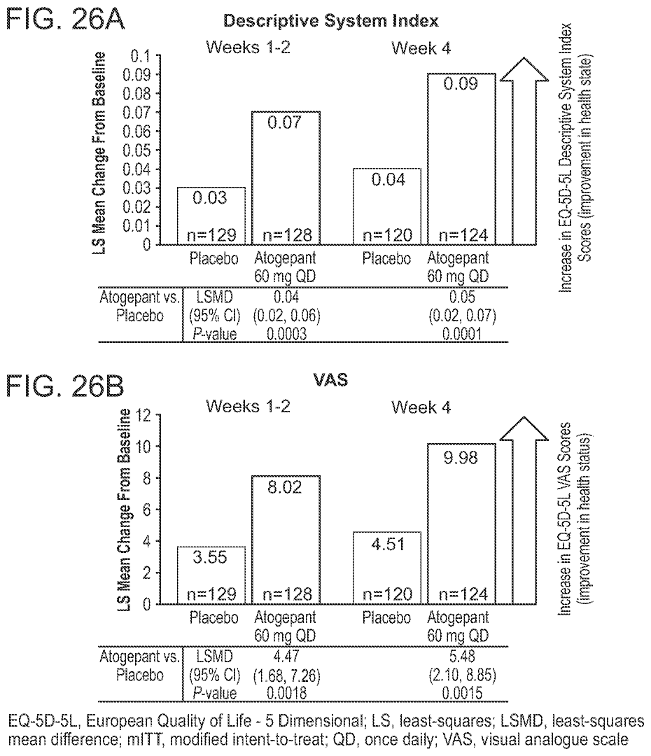

Among people with CM, atogepant significantly improved daily functioning and reduced physical impairment as early as week 1 and improved QOL within the first 2 weeks. These results demonstrate the ability of atogepant to rapidly improve function and QOL among those with CM. As disclosed herein, a total of 755 participants comprised the modified intent-to-treat population (placebo, n=246; atogepant 30 mg BID, n=253; atogepant 60 mg QD, n=256). Mean (SD) baseline values were similar between groups for the AIM-D PDA (atogepant 60 mg QD: 30.5 [18.0]; placebo 28.9 [16.7]) and AIM-D PI (atogepant 60 mg QD 27.0 [17.9]; placebo 25.1 [15.7]). Atogepant 60 mg QD demonstrated a greater improvement from baseline as early as week 1 in AIM-D PDA (least squares mean difference [LSMD]: −5.83; nominal P<0.0001) and greater reductions in PI (LSMD: −4.12; nominal P=0.0004) scores vs placebo (FIG. 25). Comparable with these results, greater improvements in AIM-D PDA and greater reductions in PI domain scores for atogepant 60 mg vs placebo were observed at weeks 2, 3, and 4. At week 1, the proportions of participants achieving clinically relevant reduction with AIM-D PDA of ≥12.5 were 29.4% for placebo and 42.9% for atogepant 60 mg QD (nominal P=0.008) and with AIM-D PI of ≥10 were 30.4% for placebo and 43.7% for atogepant 60 mg QD (nominal P=0.003). Similar AIM-D PDA and PI results were shown in the atogepant 30 mg BID group. Mean (SD) baseline values for the EQ-5D-5L descriptive system (atogepant 60 mg QD 0.76 [0.13]; placebo 0.77 [0.11]) and VAS scores (atogepant 60 mg QD 65.0 [16.4]; placebo 64.4 [15.4]) were similar between groups. Greater improvements in the mean EQ-5D-5L descriptive system (LSMD: 0.04; nominal P=0.0003) and VAS (LSMD: 4.47; nominal P=0.0018) scores were observed for atogepant 60 mg vs placebo within the first 2 weeks and consistent results were seen at week 4 (FIG. 26). Similar EQ-5D-5L results were observed in the atogepant 30 mg BID group.

In participants with chronic migraine (CM) and moderate to severe symptoms of depression as measured by 9-item Patient Health Questionnaire (PHQ−9), atogepant demonstrated a greater treatment benefit compared to placebo in migraine day reduction, responder rate, and patient-reported outcomes including the Migraine-Specific Quality of Life Questionnaire v2.1 (MSQ v2.1) and Patient Global Impression of Change (PGIC). Depression is a common comorbidity in people with migraine and is a risk factor for transformation of EM to CM. Depressive symptoms predict increases in headache-related disability and poor health-related quality of life (HRQoL) in people with migraine. Thus, trial data analysis was used to evaluate the effect of atogepant in participants with CM and baseline symptoms of moderate to severe depression. Specifically, analysis of trial data evaluated the impact of atogepant on change in monthly migraine days, ≥50% and ≥75% responder rates, change in MSQ v2.1 domain scores, and PGIC in participants with CM and moderate to severe depression symptomology. PHQ-9 is a validated, screening and diagnostic tool designed to improve the recognition rate of depression and facilitates diagnosis and treatment. A cut-off score of ≥10 was used to identify participants with moderate or severe depressive symptoms, to determine eligibility and as a continuous outcome to measure changes in depression. As disclosed hereinabove, the MSQ v2.1 is a 14-item measure of health-related quality of life evaluating impacts attributed to migraine over the past 4 weeks and comprises three domains: Role Function-Restrictive (RFR), Role Function-Preventive (RFP), and Emotional Function (EF). PGIC is a single item used to measure the subject's impression of overall change in migraine since the first dose of study intervention. Findings for the atogepant 60 mg QD dose are presented herein. Similar results were observed for the 30 BID dose (excluded for simplicity). For the endpoint change from baseline in monthly migraine days and each MSQ v2.1 domain score, the comparison between treatment groups was analyzed using a restricted maximum likelihood-based mixed model for repeated measures (MMRM). Logistic regression models were used to analyze PGIC responder at week 12 and ≥50% or 75% responder rates across 12-week double-blind treatment period. ANCOVA model was used to analyze change from baseline in PHQ-9 score at week 12.

Atogepant 60 mg QD demonstrated a greater treatment benefit over placebo in monthly migraine day reduction, proportion of participants achieving ≥50% or ≥75% responder rates, MSQ v2.1 domain scores, and PGIC in the subgroup of participants with CM and moderate to severe symptoms of depression.

Of the 778 participants enrolled, 755 (n=253 in the 30 mg BID, n=256 in the atogepant 60 mg QD and n=246 in the placebo arm) were included in the modified intent-to-treat population. Of these, 109 (42.6%) in the atogepant 60 mg QD arm and 89 (36.2%) in the placebo arm were participants with CM and symptoms of moderate to severe depression at baseline. Among participants with CM and moderate to severe depression, compared to placebo, atogepant 60 mg QD demonstrated greater reduction in migraine days over the 12-week treatment period, least squares mean differences (LSMDs): −3.04 (95% CI: −4.78, −1.29; p=0.0007) (Table 12). A larger proportion of participants on atogepant 60 mg QD achieved ≥50% (odds ratio (OR)=3.7 [95% CI: 1.86, 7.37; p=0.0002]) and ≥75% reductions (OR=13.64 [95% CI: 1.75, 106.2; p=0.01]) in monthly migraine days relative to placebo (Table 12).

TABLE 12
Monthly migraine days and responder rates across
12-week treatment period (mITT population)

| | |
|---|---|
| | Atogepant |
| Placebo | 60 mg QD |
| (n = 89) | (n = 109) |

Monthly Migraine Days

| | | |
|---|---|---|
| Baseline, mean (SD) | 19.80 | 20.14 |
| | (4.72) | (5.35) |
| Post-baseline (Month 1-3), | 15.65 | 12.90 |
| | (6.25) | (8.29) |
| mean (SD) | | |
| Change from baseline, mean | −4.14 | −7.23 |
| | (5.77) | (6.41) |
| (SD) | | |
| MMRM, LS mean change | −3.67 | −6.71 |
| | (0.69) | (0.65) |
| (SE) | | |
| LSMD vs placebo (95% CI) | | −3.04 |
| | | (−4.78, −1.29) |
| P value | | 0.0007 |

Responder Rate
≥50% Responders

| | | |
|---|---|---|
| Responders (n, %) | 16 | 46 |
| | (18.0) | (42.2) |
| Odds ratio vs placebo (95% | | 3.70 |
| | | (1.86, 7.37) |
| CI) | | |
| P value | | 0.0002 |
| ≥75% Responders | | |
| Responders (n, %) | 1 | 15 |
| | (1.1) | (13.8) |
| Odds ratio vs placebo (95% | | 13.64 |
| | | (1.75, 106.2) |
| CI) | | |
| P value | | 0.01 |

MMRM = mixed-effects model for repeated measures for change from baseline.
SD = standard deviation, SE = standard error of the least squares, CI = confidence interval, LSMD = least squares mean difference.
The population-level summary for this endpoint is the odds ratio from a logistic regression for each atogepant group relative to placebo with baseline monthly migraine days as a covariate, stratification of region, acute medication overuse, migraine prevention medication and number of failures, and treatment group as fixed factors.

Greater improvement was seen in the MSQ v2.1 domain scores at week 4 and the effect was maintained through week 12 (RFR, LSMD (95% CI): 8.77 (2.23, 15.32) [p=0.009]; RFP, LSMD (95% CI): 8.89 (2.80, 14.99) [p=0.004]; EF, LSMD (95% CI): 9.24 (2.05, 16.43) [p=0.01]) Table 13. Greater proportion of participants reported much better or very much better on the PGIC at week 12 among atogepant 60 mg QD participants compared to placebo (OR=2.52 [95% CI: 1.35, 4.70; p=0.004]). Greater reduction in PHQ-9 scores were observed at week 12 for the atogepant 60 mg QD vs placebo (LSMD (95% CI): −1.38(−2.89, 0.12) [p=0.07]) (Table 2).

TABLE 13
MSQ v2.1 domain scores at week 12 (mITT population)
MSQ v2.1

| | | Atogepant | | Atogepant | | Atogepant | PHQ-9 | |
|---|---|---|---|---|---|---|---|---|
| | Placebo | 60 mg QD | Placebo | 60 mg QD | Placebo | 60 mg QD | | Atogepant |
| | (n = 89) | (n = 109) | (n = 89) | (n = 109) | (n = 89) | (n = 109) | Placebo | 60 mg QD |
| | MSQ v2.1 RFR | | MSQ v2.1 RFP | | MSQ v2.1 EF | | (n = 89) | (n = 109) |
| Baseline, mean (SD) | 32.94 | 33.49 | 47.83 | 48.76 | 39.52 | 40.62 | | |
| | (16.20) | (15.52) | (20.93) | (22.93) | (22.01) | (26.55) | | |
| Post-baseline, mean | 51.84 | 61.53 | 63.67 | 73.71 | 61.37 | 70.79 | | |
| (SD) | (23.95) | (23.38) | (25.88) | (23.44) | (27.46) | (24.47) | | |
| Change from | 18.90 | 28.04 | 15.84 | 24.95 | 21.85 | 30.17 | | |
| baseline, mean (SD) | (22.98) | (26.37) | (19.57) | (26.94) | (27.85) | (33.01) | | |
| MMRM, LS mean | 16.78 | 25.56 | 14.01 | 22.91 | 18.95 | 28.19 | | |
| change (SE) | (2.56) | (2.43) | (2.40) | (2.27) | (2.81) | (2.66) | | |
| LSMD vs placebo | | 8.77 | | 8.89 | | 9.24 | | |
| (95% CI) | | (2.23, 15.32) | | (2.80, 14.99) | | (2.05, 16.43) | | |
| P value | | 0.009 | | 0.004 | | 0.01 | | |
| Baseline, mean (SD) | | | | | | | 14.30 | 14.41 |
| | | | | | | | (3.44) | (3.88) |
| Week 12, mean | | | | | | | 10.36 | 8.81 |
| (SD) | | | | | | | (5.59) | (6.08) |
| Change from | | | | | | | −3.93 | −5.61 |
| baseline, mean (SD) | | | | | | | (5.04) | (6.07) |
| ANCOVA, LS mean | | | | | | | −3.73 | −5.11 |
| change (SE) | | | | | | | (0.59) | (0.57) |
| LSMD vs placebo | | | | | | | | −1.38 |
| (95% CI) | | | | | | | | (−2.89, 0.12) |
| P-value | | | | | | | | 0.07 |

LS, least squares; LSMD, least squares mean difference; MMRM, mixed-effects model for repeated measures for change from baseline; SE, standard error of the least squares.
RFR—Role Function-Restrictive domain
RFP—Role Function-Preventive domain
EF—Emotional Function domain
ANCOVA for change from baseline.
PHQ-9, 9-item Patient Health Questionnaire

Similarly, in participants with chronic migraine (CM) and moderate to severe symptoms of depression, atogepant demonstrated a statistically significant treatment benefit relative to placebo in headache day reduction, moderate/severe headache days, acute medication use day reduction, and patient-reported outcomes including the Headache Impact Test 6 (HIT-6) and AIM-D. Analysis further affirmed the effect of atogepant in participants with CM and baseline symptoms of depression of moderate to severe intensity.

Analysis of trial data evaluated the impact of atogepant on headache day reduction, moderate/severe headache days, acute medication use day reduction, impacts of headache and migraine as evaluated by the HIT-6 and AIM-D, and symptoms of moderate to severe depression as measured by PHQ-9. A cut-off score of ≥10 was used to identify participants with moderate or severe depressive symptoms, to determine eligibility and as a continuous outcome to measure changes in depressive symptoms. Findings for the atogepant 60 mg QD dose are presented here. Similar results for the 30 BID dose (excluded for simplicity). For endpoints other than PHQ-9, the comparison between treatment groups were analyzed using a restricted maximum likelihood-based mixed model for repeated measures (MMRM). ANCOVA model was used to analyze PHQ-9 related endpoints.

In people with CM and moderate or severe depressive symptoms, atogepant 60 mg QD demonstrated multiple nominally significant treatment benefits over placebo in headache day reduction, moderate/severe headache day reduction, acute medication use day reduction, and the patient-reported outcomes of HIT-6 and AIM-D scores. As disclosed herein, the 778 participants enrolled, 755 (n=253 in the 30 mg BID, n=256 in the atogepant 60 mg QD and n=246 in the placebo arms) were included in the modified intent-to-treat population (mITT) population. Of these, 109 (42.6%) in the atogepant 60 mg QD arm and 89 (36.2%) in the placebo arm had PHQ-9 scores ≥10 and were included in the analysis. Compared to placebo, atogepant 60 mg QD demonstrated greater reduction in headache days, acute medication use days, and moderate/severe headache days over the 12-week treatment period (Headache days, least squares mean differences (LSMDs): −2.86 [95% CI: −4.51, −1.21; p=0.0007]; moderate/severe headache days, LSMDs: −2.68 [95% CI: −4.30, −1.06; p=0.001]); acute medication use days, LSMDs: −3.53 [95% CI: −5.15, −1.91; p<0.0001] (Table 14).

TABLE 14
Monthly headache days, acute medication use, and moderate/severe
headache days across 12-week treatment period (mITT population)

| | |
|---|---|
| | Atogepant |
| Placebo | 60 mg QD |
| (n = 89) | (n = 109) |

Monthly Headache Days

| | | |
|---|---|---|
| Baseline, mean (SD) | 21.88 | 22.18 |
| | (4.10) | (4.42) |
| Post-baseline (Month 1-3), mean (SD) | 17.73 | 15.24 |
| | (6.56) | (8.27) |
| Change from baseline, mean (SD) | −4.15 | −6.94 |
| | (4.95) | (6.33) |
| MMRM, LS mean change (SE) | −3.66 | −6.52 |
| | (0.65) | (0.62) |
| LSMD vs placebo (95% CI) | | −2.86 |
| | | (−4.51, −1.21) |
| P value | | 0.0007 |
| Monthly Moderate/Severe Headache | | |
| Days | | |
| Baseline, mean (SD) | 16.05 | 17.57 |
| | (6.12) | (5.98) |
| Post-baseline (Month 1-3), mean (SD) | 11.80 | 10.22 |
| | (6.30) | (7.95) |
| Change from baseline, mean (SD) | −4.25 | −7.34 |
| | (5.66) | (6.41) |
| MMRM, LS mean change (SE) | −4.08 | −6.77 |
| | (0.65) | (0.60) |
| LSMD vs placebo (95% CI) | | −2.68 |
| | | (−4.30, −1.06) |
| P value | | 0.001 |

Monthly Acute Medication Use Days

| | | |
|---|---|---|
| Baseline, mean (SD) | 15.22 | 17.02 |
| | (7.36) | (7.22) |
| Post-baseline (Month 1-3), mean (SD) | 11.69 | 9.51 |
| | (6.88) | (7.81) |
| Change from baseline, mean (SD) | −3.53 | −7.52 |
| | (5.73) | (6.90) |
| MMRM, LS mean change (SE) | −3.04 | −6.57 |
| | (0.67) | (0.62) |
| LSMD vs placebo (95% CI) | | −3.53 |
| | | (−5.15, −1.91) |
| P value | | <0.0001 |

MMRM = mixed-effects model for repeated measures for change from baseline. SD = standard deviation, SE = standard error of the least squares, CI = confidence interval, LSMD = least squares mean difference.

Greater reductions were observed for atogepant vs. placebo in the HIT-6 scores at week 4 and the effect was maintained through week 12, LSMDs: −3.18 (95% CI: −5.54, −0.82; p=0.008) (Table 15). Greater improvements in AIM-D PDA and greater reduction in PI domain scores across the 12-week treatment period were also observed (PDA, LSMD (95% CI): −5.91 (−9.44, −2.38) [p=0.001]; PI, LSMD (95% CI): −4.63 (−7.99, −1.28) [p=0.007]) (Table 2). Greater reduction in PHQ-9 scores were observed at week 12 for the atogepant 60 mg QD vs placebo (LSMD (95% CI): −1.38(−2.89, 0.12) [p=0.07]) (Table 15).

TABLE 15
HIT-6 and PHQ-9 scores at week 12, and AIM-D PDA and PI domain
scores across the 12-week treatment period (mITT population)

| | |
|---|---|
| | Atogepant |
| Placebo | 60 mg QD |
| (n = 89) | (n = 109) |

HIT-6

| | | |
|---|---|---|
| Baseline, mean (SD) | 66.31 | 66.41 |
| | (3.76) | (4.30) |
| Post-baseline, mean (SD) | 60.86 | 57.68 |
| | (7.51) | (9.00) |
| Change from baseline, mean (SD) | −5.46 | −8.73 |
| | (7.27) | (8.88) |
| MMRM, LS mean change (SE) | −5.18 | −8.37 |
| | (0.93) | (0.87) |
| LSMD vs placebo (95% CI) | | −3.18 |
| | | (−5.54, −0.82) |
| P value | | 0.008 |

AIM-D PDA

| | | |
|---|---|---|
| Baseline, mean (SD) | 35.69 | 38.38 |
| | (14.91) | (17.22) |
| Months 1-3, mean (SD) | 26.94 | 22.29 |
| | (13.87) | (18.05) |
| Change from baseline, mean (SD) | −8.76 | −16.09 |
| | (12.75) | (14.20) |
| MMRM, LS mean change (SE) | −7.59 | −13.50 |
| | (1.40) | (1.30) |
| LSMD vs placebo (95% CI) | | −5.91 |
| | | (−9.44, −2.38) |
| P-value | | 0.001 |

AIM-D PI

| | | |
|---|---|---|
| Baseline, mean (SD) | 31.08 | 33.91 |
| | (14.25) | (17.44) |
| Months 1-3, mean (SD) | 23.41 | 20.07 |
| | (12.48) | (17.29) |
| Change from baseline, mean (SD) | −7.66 | −13.84 |
| | (12.87) | (13.76) |
| MMRM, LS mean change (SE) | −7.04 | −11.68 |
| | (1.33) | (1.24) |
| LSMD vs placebo (95% CI) | | −4.63 |
| | | (−7.99, −1.28) |
| P-value | | 0.007 |
| PHQ-9 | | |
| Baseline, mean (SD) | 14.30 | 14.41 |
| | (3.44) | (3.88) |
| Week 12, mean (SD) | 10.36 | 8.81 |
| | (5.59) | (6.08) |
| Change from baseline, mean (SD) | −3.93 | −5.61 |
| | (5.04) | (6.07) |
| ANCOVA, LS mean change (SE) | −3.73 | −5.11 |
| | (0.59) | (0.57) |
| LSMD vs placebo (95% CI) | | −1.38 |
| | | (−2.89, 0.12) |
| P-value | | 0.07 |

HIT-6, 6-item Headache Impact Test; AIM-D, Activity Impairment in Migraine Diary; PDA, Performance of Daily Activities; PI, Physical Impairment; LS, least squares; LSMD, least squares mean difference; MMRM, mixed-effects model for repeated measures for change from baseline; SE, standard error of the least squares. ANCOVA for change from baseline. PHQ-9, 9-item Patient Health Questionnaire.

1. A method for the preventive treatment of migraine, wherein the migraine is chronic migraine, the method comprising administering atogepant or a pharmaceutically acceptable salt thereof in an amount of 60 mg once daily or 30 mg twice daily to a patient in need of treatment for migraine.

2. The method according to claim 1, wherein treatment with atogepant achieves a reduction from baseline in mean monthly migraine days of at least about 6.7 days.

3. The method according to claim 1, wherein treatment with atogepant achieves a reduction from baseline in mean monthly migraine days of at least about 1.8 days.

4. The method according to claim 1, wherein treatment with atogepant achieves a reduction from baseline in mean monthly migraine days of at least about 7.3 days.

5. The method according to claim 1, wherein treatment with atogepant achieves a reduction from baseline in mean monthly migraine days of at least about 7.4 days.

6. The method according to claim 1, wherein administration of atogepant achieves a statistically significant change in mean monthly migraine days from baseline to week 12 of said treatment.

7. The method according to claim 1, wherein administration of atogepant achieves a statistically significant change in mean monthly headache days from baseline to week 12 of said treatment.

8. The method according to claim 1, wherein administration of atogepant achieves a statistically significant change in mean monthly acute medication use days from baseline to week 12 of said treatment.

9. The method according to claim 1, wherein administration of atogepant achieves a statistically significant change from baseline to week 12 in the proportion of patients with ≥50% reduction in 3-month average monthly migraine days.

10. The method according to claim 1, wherein administration of atogepant achieves a statistically significant change in MSQ v2.1 Role Function-Restrictive Domain Score in said patients from baseline to week 12.

11. The method according to claim 1, wherein administration of atogepant achieves a statistically significant change in the Mean Monthly Performance of Daily Activities Domain Score of the AIM-D in said patients from baseline to week 12.

12. The method according to claim 1, wherein administration of atogepant achieves a statistically significant change in the mean Monthly Physical Impairment Domain score of the AIM-D in said patients from baseline to week 12.

13. The method according to claim 1, wherein administration of atogepant achieves a statistically significant change from baseline to week 12 in the HIT-6 Total Score in said patients.

14. A method of statistically significant treatment of migraine in patients in need thereof, wherein the migraine is chronic migraine, comprising administering to each patient atogepant or a pharmaceutically acceptable salt thereof in an amount of 30 mg twice daily or 60 mg once daily, wherein said treatment achieves a statistically significant least square mean difference in monthly migraine days in said patients from baseline to week 12 of said treatment as compared to placebo.

15. The method according to claim 14, wherein the least square mean difference in monthly migraine days is at least about −1.6 as compared to placebo.

16. The method according to claim 14, wherein the least square mean difference in monthly migraine days is at least about −1.8 as compared to placebo.

17. The method according to claim 14, wherein the least square mean difference in monthly migraine days is at least about −2.2 as compared to placebo.

18. The method according to claim 14, wherein the least square mean difference in monthly migraine days is at least about −2.4 as compared to placebo.
